(19) United States
(12) Patent Application Publication
(10) Pub. No.: US 2022/0347337 A1
(43) Pub. Date: Nov. 3, 2022

(54) APPARATUS, METHOD AND SYSTEM TO PROVIDE SAFE MEETING SPACES FOR FACE-TO-FACE COMMUNICATION AND IN-PERSON EVENTS

(71) Applicant: Daniel NORRIS, London (GB)

(72) Inventor: Daniel NORRIS, London (GB)

(21) Appl. No.: 17/726,603

(22) Filed: Apr. 22, 2022

Related U.S. Application Data
(60) Provisional application No. 63/180,755, filed on Apr. 28, 2021.
(60) Provisional application No. 63/211,720, filed on Jun. 17, 2021.
(60) Provisional application No. 63/296,538, filed on Jan. 5, 2022.

(51) Int. Cl. A61L 9/20; A61L 2/26; A61L 2/06; A61L 2/03; A61L 2/10; A61L 2/08; F24F 8/22; G06Q 10/10

(52) U.S. Cl. CPC A61L 9/20; A61L 2/26; A61L 2/06; A61L 2/03; A61L 2/10; A61L 2/085; A61L 2/084; F24F 8/22; G06Q 10/10; A61L 2202/25; A61L 2209/16

(57) ABSTRACT

The field of this invention is technology that supports and enhances natural face-to-face communication. Throughout the recent Pandemic, people have been adversely affected by the loss of real face-to-face human contact. The invention provides one or more safe ‘meeting spaces’ through which persons can talk to each other normally, without the risk of airborne pathogens passing between them. The meeting space of the invention offers no physical barrier such as glass, which would reduce the quality of communication and diminish the user experience. Persons may see and hear each other through the meeting space but pathogens cannot substantially pass through it. Various ways are provided that substantially destroy/inactivate/remove pathogens from the meeting space(s). Some embodiments are sustainable, carbon neutral and solar powered. Others are highly energy efficient. Further refinements create a pleasant environment for conversation and enhance the user experience.

This application claims priority of U.S. Provisional Application No. 63/180,755 filed on Apr. 28, 2021, and U.S. Provisional Application No. 63/211,720 filed on Jun. 17, 2021, and U.S. Provisional Application No. 63/296,538 filed on Jan. 5, 2022 all of which are incorporated herein in their entirety.

### BACKGROUND—FIELD OF THE INVENTION

The field of this invention is technology that supports and enhances natural face-to-face communication. In some preferred embodiments, the primary field of the invention is Communication.

### BACKGROUND

Most communication systems eliminate real-life face-to-face interaction and replace it with an inferior digital version. In some embodiments, the invention promotes and supports natural conversation by making it safe for users. In some embodiments, the invention works synergistically with other technologies that facilitate face-to-face communication such as U.S. Pat. No. 7,111,244 and United States Pre-Grant Publication No. 2020/0334765

Throughout the current Covid-19 Pandemic, people have felt isolated and have been adversely affected by the loss of real face-to-face human contact. Digital alternatives such as Zoom are second best, but they lack the power and the immediacy of natural face-to-face conversation. This invention enables users to talk to each other normally, while reducing or eliminating the risk of infection.

The standard apparatus to reduce the spread of pathogens during face-to-face interaction is a glass screen. However, the screen reduces the quality of communication, and it creates all the intimacy of a prison visit! Furthermore, persons on the same side of a screen are at increased risk of infection as it restricts airflow and ventilation.

The other standard approach to making face-to-face human contact safe is to replace it with a digital simulation such as Zoom. Natural face-to-face conversation has many advantages over the various online alternatives. Humans have adapted and evolved to communicate by talking in three-dimensional space. Online communication takes place on a flat 2-dimensional screen, there is significant loss of quality and an inferior user experience. As a result of the Covid-19 pandemic, in-person events are increasingly seen as the gold-standard to which virtual online events are compared.

The invention solves an important problem, particularly in the light of the Pandemic. People have experienced significant social isolation. The invention does not simply limit the spread of harmful pathogens, in some embodiments it facilitates safe social interaction through natural face-to-face communication. As such, the invention meets a basic human need.

In part, the invention lies in the formulation of the problem to be solved, particularly the need for safe human contact and for natural face-to-face conversation, without intrusive barriers between persons.

### SUMMARY OF THE INVENTION

In some embodiments, the invention provides a system that creates one or more safe (pathogen-free) meeting space(s) for two or more persons. Sound may pass through the meeting space(s) but pathogens may not by the system fully or substantially destroying, inactivating and/or removing pathogens from the meeting space(s). Persons are able to see and hear each other without impediment—there is no see-through barrier, e.g., glass. As such, persons are able to communicate safely through natural face-to-face conversation.

In some embodiments, the invention provides at least one safe ‘meeting space’ through which at least two persons can talk to each other normally, without the risk of airborne pathogens passing between them. The at least one meeting space of some embodiments of the invention offers no physical barrier such as glass, which would reduce the quality of communication and diminish the user experience. At least two persons may see and hear each other through the at least one meeting space but pathogens cannot substantially pass through it and/or pathogens are completely or substantially prevented from passing through the at least one meeting space. Various means are used to substantially and/or completely destroy/inactivate/remove pathogens from the at least one meeting space.

In some embodiments, the invention is a system providing one or more safe meeting spaces for two or more persons wherein:
a) the system provides a central meeting space and person locations outside the central meeting space for persons using the central meeting space, with no barriers between the central meeting space and the person locations such that persons in the person locations can communicate with each other through the central meeting space,
b) outside the central meeting space and between adjacent person locations, are provided adjacent person meeting spaces or partitions,
c) the system is configured such that substantially all pathways within the system for the possible spread of pathogens between the person locations pass through at least one of the meeting spaces,
d) means are provided to make the meeting space(s) sterile or to substantially reduce the number of pathogens in the meeting spaces(s),
e) the system is configured to allow for persons in the person locations using the same meeting space(s) to hear each other through the meeting spaces(s) and for at least two persons using the same meeting space(s) to see each other through the meeting spaces(s).

In some embodiments, the invention is a system providing more than one safe meeting space for two or more persons wherein:
a) the system provides more than one meeting space and person locations outside the meeting spaces for persons using the meeting spaces, with no barriers between the meeting space(s) and the person locations such that persons in the person locations using the same meeting space(s) can communicate with each other through the meeting space(s),
b) the system is configured such that substantially all pathways within the system for the possible spread of pathogens between persons using the same meeting spaces pass through at least one of the meeting spaces, or the system is configured such that substantially all pathways within the system for the possible spread of pathogens between the person locations pass through at least one of the meeting spaces,
c) means are provided to make the meeting space(s) sterile or to substantially reduce the number of pathogens in the meeting spaces(s),
d) the system is configured to allow for persons in the person locations using the same meeting space(s) to hear each other through the meeting spaces(s) and for at least two persons using the same meeting space(s) to see each other through the meeting space(s).

In some embodiments, the means comprise one or more of the following:
a) at least one heat source and/or heated air produced by a heat source and/or a thermal updraft;
b) ultraviolet and/or infrared and/or concentrated solar radiation;
c) at least one electrostatic attractor and/or at least one kind of airborne ion;
d) at least one anti-pathogenic agent;
e) at least one airflow.

The use of multiple and/or complementary means in some embodiments, increases the efficiency of the means in destroying, inactivating and/or removing pathogens from the meeting space(s).

Further features of some embodiments, make the invention less obtrusive and/or safer for persons. In some embodiments, adjacent persons using a central meeting space are provided with adjacent person meeting space(s) instead of partitions. In some of embodiments, meeting spaces that are close to persons, including adjacent person meeting spaces, use means that are not harmful to persons. Safer means include laminar air flows and air heated to sufficient temperature to substantially destroy pathogens without being hot enough to harm persons.

Further features of some embodiments enhance the user experience and/or increase the functionality of the invention for the facilitation of conversation and/or discussion.

An additional feature of some embodiments is the use of the means to create a pleasing visual focus for discussion and/or to create a stimulating environment for conversation. A further feature of some embodiments is the use of the means to displaying subjects for discussion in the meeting space(s).

Some embodiments of the invention are energy efficient, carbon neutral and sustainable. Some embodiments utilise solar energy. Some solar embodiments of the invention separate sunlight into UV, IR and visible light to provide multiple and/or complementary means to destroy, inactivate and/or to remove pathogens.

Buildings need to be heated so it is highly efficient to use the same energy to create safe meeting spaces. A further feature of some embodiments is the use of heat energy and/or heated air from the meeting space(s) to heat buildings or outdoor spaces. The dual efficiency of using the same heat energy to create safe meeting spaces and also to provide heating, is an important advantage of some embodiments the invention. Further additional features increase energy efficiency, such as recycling heat energy from the meeting space back into the meeting space.

Some embodiments provide multiple meeting spaces/multiple central meeting spaces and means are provided to prevent pathogens passing between persons using different meeting spaces.

In some embodiments, the meeting space(s) have extraction means and are at negative pressure in relation to the person locations. In these embodiments, the meeting space(s) act as wells for the extraction of pathogens from the space in which they are located. This is another important advantage of the invention.

In some embodiments, the heat energy that provides the means is also used to provide warm clean (pathogen-free) air, which is supplied to the zone surrounding person locations, increasing the positive pressure around person locations and increasing the flow of air into person locations, thereby increasing the flow of pathogens from the person locations into the meeting space(s).

In some embodiments, cool clean air is supplied to the zone surrounding person locations, increasing the positive pressure around person locations. This sort of embodiment reduces the flow of pathogens from person locations into the space where the meeting space(s) are located. This sort of embodiment reduces the spread of pathogens between persons using different meeting spaces.

### BRIEF DESCRIPTION OF THE DRAWINGS

FIG. 1 shows a possible embodiment with a central meeting space surrounded by person locations. Partitions are provided between adjacent person locations so that pathogens cannot pass between persons in adjacent person locations using the central meeting space. This possible embodiment is configured so that substantially all pathways for the possible spread of pathogens pass through the central meeting space.

FIG. 2 shows a possible embodiment with multiple complimentary means to destroy, inactivate and/or to remove pathogens from the meeting space, including: fire/flames, thermal updraft and extraction means, an IR reflector/concentrator and an electrostatic attractor. An additional feature is the use of the means to display subjects for discussion in the meeting space.

FIG. 3 illustrates an additional feature, the use of sound reflectors to enhance communication between persons using the meeting space.

FIG. 4 illustrates the use of solar energy to provide multiple complementary means to substantially destroy/inactivate and/or remove pathogens from the meeting space. In some embodiments, sunlight is separated into UV and IR to provide complimentary means. An additional feature is the storage of solar energy to provide means when sunlight is not available.

FIG. 5 illustrates pathogen extraction and sterilization means.

FIG. 6 is a plan view of a possible configuration for solar sterilization of a central meeting space.

FIG. 7 illustrates possible embodiment(s), separating sunlight into UV, IR and visible light, providing multiple complementary means for sterilisation and pathogen extraction. Additional features—the use of UV and visible light to create pleasing visual effects to serve as a focus for conversation and also to display subjects for discussion in the meeting space are also illustrated.

FIG. 8 is a plan view of another possible embodiment of the invention with a central meeting space and meeting spaces for adjacent persons using the central meeting space (adjacent person meeting spaces). The means used in the central meeting space is the same as the means used in the adjacent person spaces.

FIG. 9 is a plan view of a variation of the possible embodiment of the invention of FIG. 8, wherein the means used in the adjacent person meeting spaces is different from the means used in the central meeting space. The means used in the adjacent person spaces is not harmful to persons.

FIGS. 10a and 10b are plan and side views of another possible embodiment of the invention providing more than one meeting space. This embodiment is configured such that substantially all pathways within the system for the possible spread of pathogens between the person locations pass through at least one of the meeting spaces.

FIG. 11 illustrates additional features of the invention that increase its energy efficiency whereby heated air from the meeting space(s) is used to heat buildings and/or is recycled back into the meeting space.

FIG. 12 illustrates additional features of the invention that increase its energy efficiency whereby heated air from the meeting space(s) is used to provide warm pathogen-free air to heat buildings and/or is recycled back into the meeting space.

FIG. 13 illustrates additional features of the invention that increase its efficiency whereby heat energy from the meeting space(s) is used to provide warm pathogen-free air to heat buildings and/or is supplied to persons in person locations increasing the flow of pathogens into the meeting space(s) to extract them.

FIG. 14 illustrates additional features of the invention that increase its efficiency whereby pathogens are extracted from the space in which the system is located and heat energy from the meeting space(s) is used to provide warm pathogen-free air to heat buildings and/or is supplied to persons in person locations increasing the flow of pathogens into the meeting space(s) to extract them.

FIG. 15 illustrates a feature that increases energy efficiency suitable for air-conditioned buildings whereby pathogens are extracted from the space in which the system is located and cool pathogen-free air is provided to cool buildings and/or is supplied to persons in person locations increasing the flow of pathogens into the meeting space(s) to extract them.

FIG. 16 is a plan view of a possible embodiment that comprises a plurality of central meeting spaces. Means are provided to prevent pathogens passing between persons using different meeting spaces.

### DETAILED DESCRIPTION OF THE INVENTION

The invention solves an important problem, particularly in the light of the Covid-19 Pandemic. People have experienced significant social isolation. The invention does not simply limit the spread of harmful pathogens, in some embodiments it facilitates safe social interaction through natural face-to-face communication. As such, the invention meets a basic human need.

In part, the invention lies in the formulation of the problem to be solved, particularly the need for safe human contact and for natural face-to-face conversation, without intrusive barriers between persons.

An important advantage of the invention over electronic communication systems is that it supports the best possible form of communication—which is face-to-face and in-person. Natural face-to-face communication is always of a much higher quality than any digital or online alternatives—which at best provide a simulation of it.

In some embodiments, the invention provides a system that creates one or more safe (pathogen-free) meeting space(s) for two or more persons. Sound may pass through the meeting space(s) but pathogens may not by the system fully or substantially destroying, inactivating and/or removing pathogens from the meeting space(s). Persons are able to see and hear each other without impediment—there is no see-through barrier, e.g., glass. As such, persons are able to communicate safely through natural face-to-face conversation.

In some embodiments, the invention provides at least one safe ‘meeting space’ through which at least two persons can talk to each other normally, without the risk of airborne pathogens passing between them. The at least one meeting space of some embodiments of the invention offers no physical barrier such as glass, which would reduce the quality of communication and diminish the user experience. At least two persons may see and hear each other through the at least one meeting space but pathogens cannot substantially pass through it and/or pathogens are completely or substantially prevented from passing through the at least one meeting space.

These diagrams and drawings discussed below serve only to illustrate aspects of the invention, which is not limited by them. It should be understood that the numbers used in the lists of features of the invention correspond to the numbers shown in the drawings.

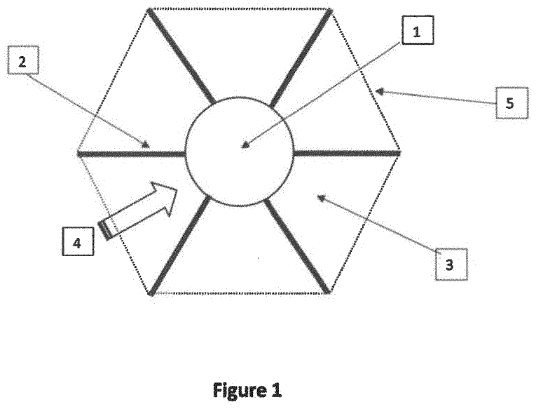

FIG. 1

A plan view of a possible embodiment is shown in FIG. 1 with the following features.
1. Central meeting space with sterilization/pathogen extraction means. The extraction means may create an upwards airflow taking pathogens away from persons.
2. Partition between adjacent persons using the central meeting space
3. Enclosure for participants (person locations)
4. Fresh air inflow
5. Barrier/partial barrier to wind or to external air movements, which may disrupt airflows.

This possible embodiment provides a central meeting space 1 and person locations 3 outside the central meeting space for persons using the central meeting space, with no barriers between the central meeting space and the person locations such that persons in the person locations can communicate with each other through the central meeting space. Partition(s) 2 prevent pathogens passing between adjacent persons using the central meeting space 1. The system is configured such that substantially all pathways within the system for the possible spread of pathogens between the person locations 3 pass through the central meeting space 1. The means make the central meeting space sterile or substantially reduce the number of pathogens in the meeting space.

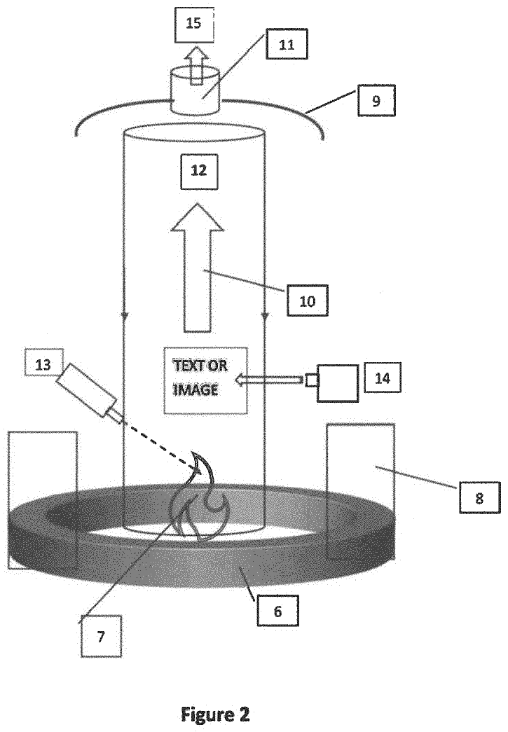

FIG. 2

FIG. 2 shows a possible embodiment with multiple complimentary means to destroy, inactivate and/or to remove pathogens from the meeting space, including: fire/flames, thermal updraft and extraction means, an IR reflector/concentrator and an electrostatic attractor. An additional feature is the use of the means to display subjects for discussion in the meeting space. Features shown in FIG. 2 include the following:
6. Low level circular seating;
7. Fire/flame;
8. Partition between participants—two are shown but numbers may be variable;
9. Concave IR reflector and charged electrostatic attractor;
10. Up-flow of air and pathogens created by convection and electrostatic attractor 9;
11. Vent out;
12. IR sterilisation beam reflected down by concave reflector 9;
13. Optional scanning laser to create text/images in fire/flames;
14. Optional LCD projector to create text/images in fire/flames or smoke; and
15. Airflow and pathogens out.

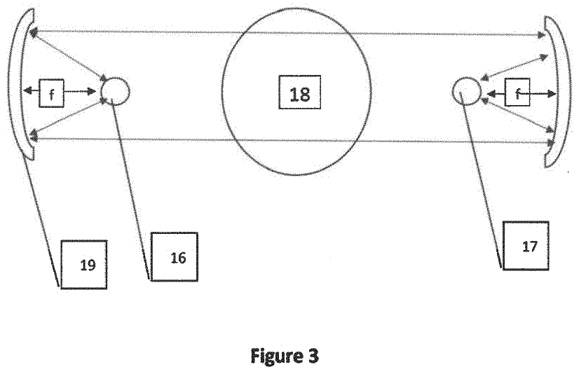

FIG. 3

FIG. 3 is a plan view showing the use of sound reflectors to direct sound waves into and out from the central meeting space. This feature is particularly suitable for sterilisation/extraction means that attenuate and dampen sound—such as fires. Sterilization/extraction means that attenuate sound, would also mitigate the risk of echo reverberation.

Features shown in FIG. 3 include the following:
16. Person 1, head is typically at focal point of sound reflector;
17. Person 2, head is typically at focal point of sound reflector;
18. Central meeting space with sterilisation means and/or means to extract pathogens; and
19. Concave reflector, optionally parabolic, reflecting sound into the central meeting space and out towards participants.

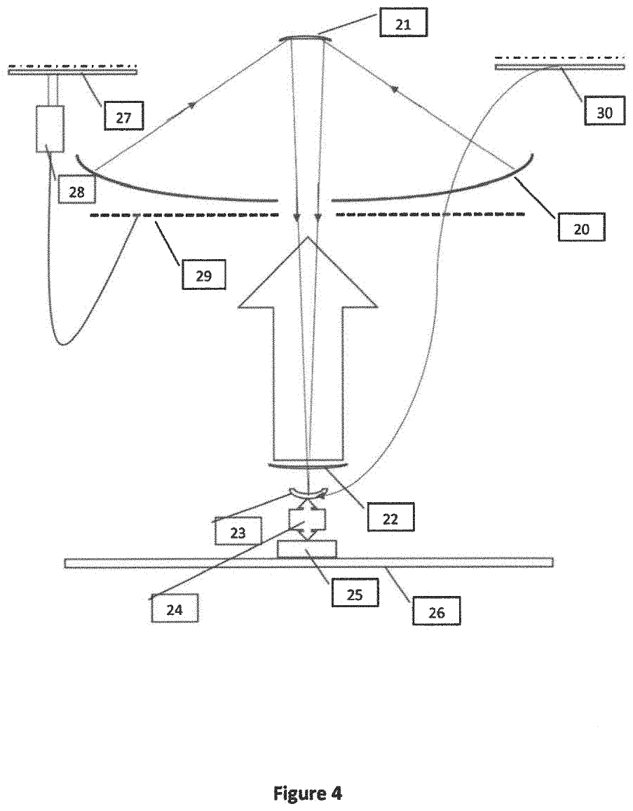

FIG. 4

FIG. 4 illustrates the use of solar power in some solar powered embodiments, to provide solar sterilization means, solar thermal pathogen extraction means, and additionally electrostatic pathogen extraction means. Optionally, with solar tracking means.

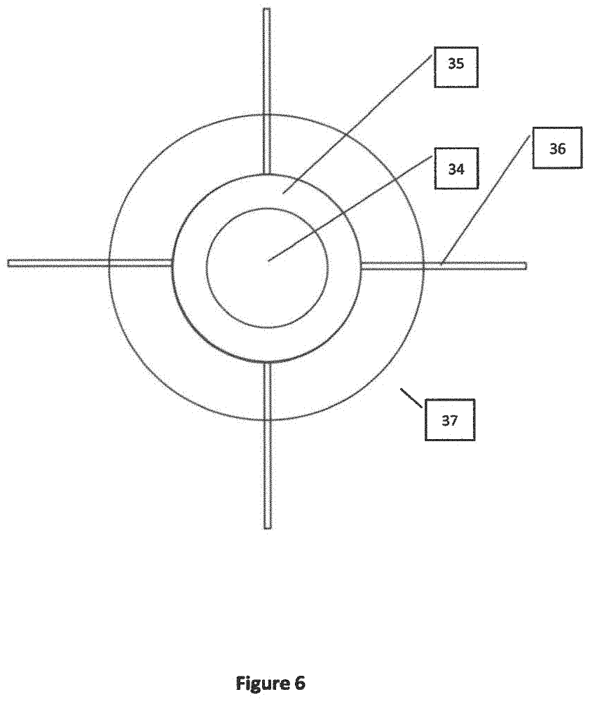

Features shown in FIG. 4 include the following.
20. Main solar reflector, typically polished aluminium and parabolic
21. Secondary reflector/concentrator typically polished aluminium and parabolic.
22. UV reflector, ideally transparent to IR and visible light. Increases intensity of UV sterilization beam shown above, see 12 in FIG. 2. The reflected UV beam may be focused into the configuration shown in FIG. 6. Alternatively, it may be used to generate static electricity by the photo-electric effect (using 27 or 30) to charge 23 (or 29). Additionally, the UV may be used to generate Ozone.
23. Heat absorbing target, creates thermal up-flow. May contain antipathogenic agent which is vapourised. Particles of antipathogenic agent may be ionised (e.g., negatively) and attracted upwards to electrostatic attractor 29 (which may be positively charged).
24. Optional heat exchanger allowing heat to flow from target 23 to heat storage unit 25.
25. Optional heat storage unit for night-time use.
26. Supporting surface optionally table or ground.
27. Photovoltaic or photoelectric array connected to alternator/transformer/rectifier unit 28 to produce positive charge for electrostatic attractor 29.
28. Alternator/transformer/rectifier to produce positive charge for electrostatic attractor 29.
29. Electrostatic attractor (here positively charged).
30. Photo-electric array producing negative charge by the photoelectric affect. Connected to heat target 23 to ionize/vaporize an antipathogenic agent.

Note in some embodiments, the attractor may be negatively charged and the evaporator/ionizer may be positively charged. Alternatively, either one may be earth grounded.

In some embodiments, sunlight is separated into UV and IR to provide complimentary means. An additional feature is the storage of solar energy to provide means when sunlight is not available.

In some embodiments, the heat absorbing target 23 is replaced by an ozone generator. Ozone is known to be anti-pathogenic and it will also bind to airborne pathogens. Ozone is harmful in high concentrations but it is drawn away from participants by the attractor 29. Additionally, the ozone will give airborne pathogens a negative charge and so they will be strongly drawn to the positive attractor 29.

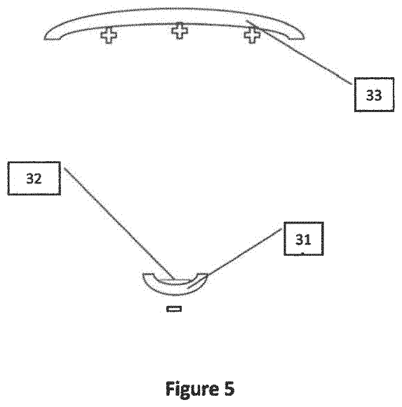

FIG. 5

FIG. 5 shows an example of pathogen extraction and sterilization means for indoor and outdoor spaces. The apparatus and method shown here may be used to remove/destroy airborne pathogens from a meeting space as described here. It may be used to remove/destroy airborne pathogens from any space such as a room.

Features shown in FIG. 5 include the following.
31. Negatively charged ioniser—containing antipathogenic agent 32 and/or an agent that binds to airborne pathogens (which are neutral) giving them a net negative charge. Negatively charged pathogens are then drawn to the positive electrostatic attractor 33. The ioniser may be heated to vaporize the agent 32 or ultrasound may be used for the same purpose. In some embodiments, 31 is an ozone generator which ionises air.
32. Antipathogenic agent and/or agent that binds to airborne pathogens when ionised.
33. Positively charged electrostatic attractor. Neutral airborne pathogens are drawn to the attractor as are pathogens bound to the negatively charged agent 32. The attractor may be heated to a sufficient temperature to destroy pathogens.

As noted above, 31 an ozone generator. Ozone is known to be anti-pathogenic and it will also bind to airborne pathogens. Ozone is harmful in high concentrations but it is drawn away from participants by the attractor 33. Additionally. the ozone will give pathogens a negative charge and so they will be drawn to the positive attractor 33.

FIG. 6

FIG. 6 shows a plan view of a possible configuration for solar sterilisation of a central meeting area and features of this configuration include the following.
34. Central meeting area—e.g., center of table.
35. Solar beam, hollow tube of sunlight or solar UV. The solar beam is a circular wall around the perimeter of the central meeting area, through which pathogens must pass twice to travel from one participant to another. Focusing sunlight or solar UV into a wall around the perimeter increases its intensity, as compared to a beam that covers the entire central area. This may increase its ability to destroy pathogens.
36. Partition between persons in person locations 37.
37. Person locations.

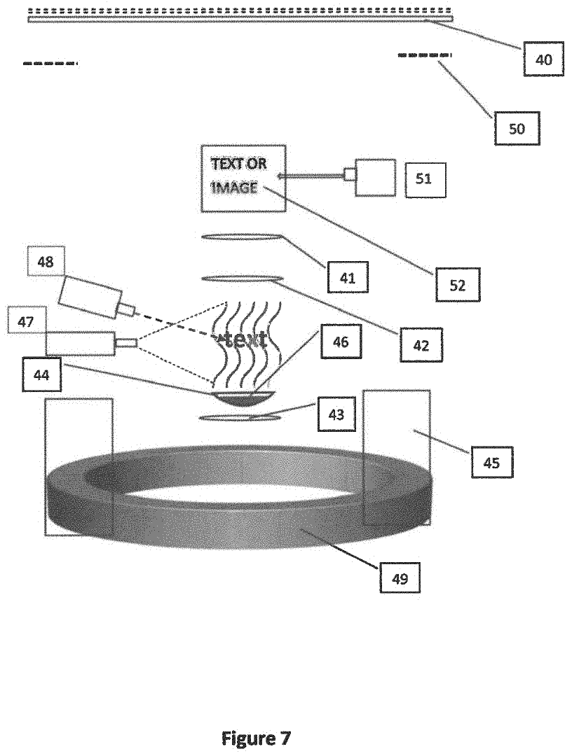

FIG. 7

FIG. 7 illustrates a possible embodiment(s), separating sunlight into UV, IR and visible light, providing multiple complementary means for sterilisation and pathogen extraction. Illustrating additional features—the use of UV and visible light to create pleasing visual effects to serve as a focus for conversation and to display subjects for discussion in the meeting space. Features shown in FIG. 7 include the following.
40. Fresnel lens or composite Fresnel lens, or compound Fresnel, providing different focal points for solar UV, solar IR and visible light. Solar tracking means may be provided which will significantly reduce the size of the focal zones for each component of sunlight.
41. Focal zone (or focal point) for UV. The UV component may be reflected away using a reflector that is transparent to IR and visible light as previously shown. The UV may be used for any one or more of the following:
   a) Solar UV may be used to generate static energy by the photoelectric effect, which may be used to ionise an agent 46 contained in a heat absorbing target 44. Or to charge electrostatic attractor ring 50.
   b) To provide a UV sterilisation beam, optionally as a hollow ring-shaped beam in the configuration of FIG. 6.
   c) Solar UV may be used to illuminate a transparent fluorescent display screen 52 (such as LCD)
   d) UV may be used by an LCD projector 51 and projected onto display screen 52 which is florescent and optionally transparent.
   e) UV may be used by projector 47 to cause a vapourised fluorescent agent 46 to glow.
   f) Solar UV may be used by projector 48 (such as an LCD projector) to project an image or text onto a vapourised agent which is florescent. Alternatively, the vapourised agent may glow when ionised and glow in a different colour when struck by UV.
   g) Solar UV may be used to generate ozone as an antipathogenic agent.
42. Focal point/zone for visible sunlight. The visible light may be converted to IR by the greenhouse effect, to increase the temperature of the heat absorbing target 44. The visible light component may be reflected away using a reflector that is transparent to IR. The visible light may be used for any one or more of the following display means.
   a) To illuminate an LCD screen 52, which is optionally semi-mirrored and transparent.
   b) For LCD projector 51 to produce an image on screen 52, which is optionally semi-mirrored and transparent.
   c) By projector 48 to produce an image or text in vapour.
43. Focal point/zone for IR. Solar IR may be used to heat the heat absorbent target 44, to produce a thermal up-flow and/or to vaporize agent 46 held by the heat absorbing target 44.
44. Heat absorbing target, typically black and optionally concave/crucible. The heat absorbing target is shaped to contain the agent 46 for vaporization. It may be given a negative electrostatic charge to ionize the agent 46. This electrostatic charge may optionally be produced by solar UV and the photoelectric effect.
45. Screen/barrier to safely separate users on seating 49.
46. Agent or mixture of agents which may have one or more of the following properties:
   a) It is antipathogenic when vaporized;
   b) When vaporized, it binds to airborne pathogens;
   c) When vaporized, and/or ionised it binds to airborne pathogens;
   d) When vaporized, and ionised it may glow/emit light;
   e) When vaporised, it emits light when illuminated by UV;
   f) When vaporized/ionised, it glows/emits light;
   g) When vaporized/ionised, it glows/emits light and additionally emits light of a different colour when illuminated by UV;
   h) When ionised, it binds to airborne pathogens and both together are attracted up towards electrostatic attractor ring 50; and
   i) When vaporised and/or ionized, and/or exposed to UV, it produces ozone.
47. Projector as previously described, optionally an LCD projector and/or a UV LCD projector.
48. Projector as previously described that produces images on vapour and/or produces images in contrasting colours if the vapour is glowing/emitting light. Alternatively, a scanning laser that produces images on vapour and/or produces images in contrasting colours if the vapour is glowing/emitting light could be provided.
49. Low level circular seating, typically of more than 2 metres diameter, for safe social distancing.
50. Electrostatic attractor ring, draws airborne pathogens up towards it. The ring may be charged by static produced by solar UV and the photoelectric effect.
51. Projector as previously described.
52. Display screen or OLED or LCD display screen, optionally and preferably transparent. Alternatively, a screen could be used onto which images/text are projected by 51. The screen onto which images/text are projected is optionally and preferably transparent or semi-mirrored and transparent.

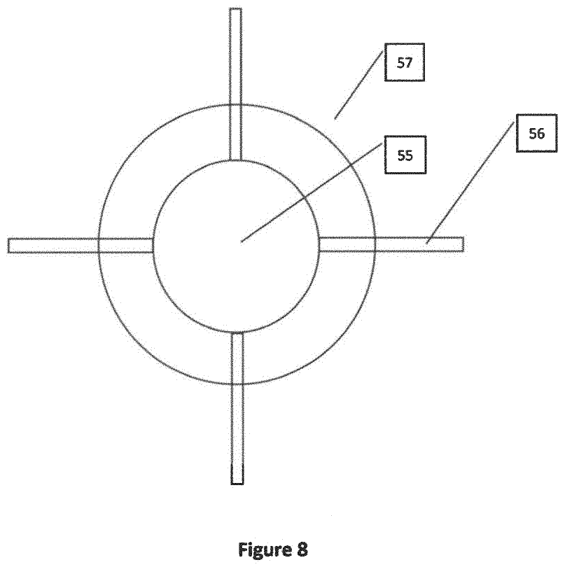

FIG. 8

FIG. 8 is a plan view of another embodiment of the invention with a central meeting space 55 and meeting spaces 56 for adjacent persons (adjacent person meeting spaces) using the central meeting space 55. The central sterilization/extraction means, that is designed for the central meeting space 55 are extended into the adjacent persons meeting space 56 to provide a non-physical barrier between adjacent persons through which pathogens cannot pass. The adjacent person meeting space 56 serves as a meeting space for adjacent persons, which contrasts with the central meeting space that serves as a meeting space for persons across the central meeting space. Airflows and lamina airflows going directly upwards are particularly safe as a barrier between participants. Participants are in separate chambers 57 (person locations), separated by sterilization means associated with the spaces 55 and 56 and the spaces 55 and 56 themselves through which pathogens cannot pass. In this embodiment, the means used to make the central meeting space safe is the same as that used to make the space between adjacent persons safe, subject to the exclusion of any of the means disclosed for the meeting space creating a danger for the adjacent persons. This sort of embodiment provides more than one meeting space between persons.

This possible embodiment provides a central meeting space 55 and person locations 57 outside the central meeting space for persons using the central meeting space, with no barriers between the central meeting space and the person locations such that persons in the person locations can communicate with each other through the central meeting space. Outside the central meeting space and between adjacent person locations, are provided adjacent person meeting spaces 56. This embodiment is configured such that substantially all pathways within the system for the possible spread of pathogens between the person locations pass through at least one of the meeting spaces. Means are provided to make the meeting space(s) sterile or to substantially reduce the number of pathogens in the meeting spaces(s). Persons in the person locations using the same meeting space(s) can hear each other through the meeting spaces(s) and persons using the same meeting space(s) can see each other through the meeting spaces(s).

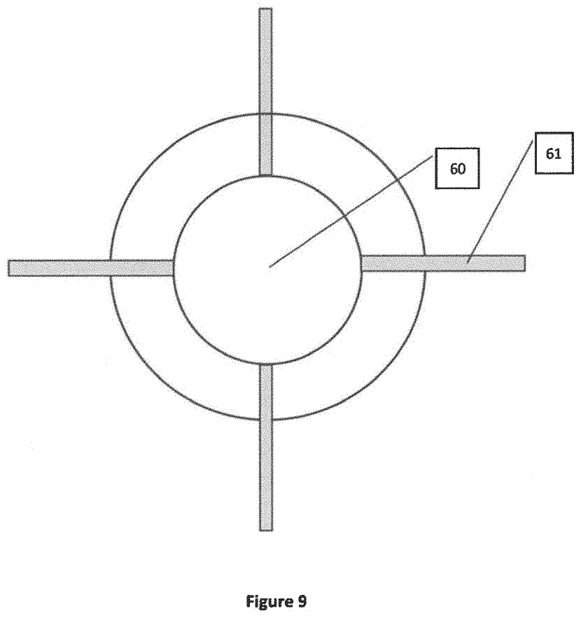

FIG. 9

FIG. 9 is a plan view of a variation of the possible embodiment of the invention of FIG. 8. In this embodiment, the central sterilization/extraction means associated with the central meeting space 60 is different as compared to sterilization/extraction means associated with the adjacent persons meeting space 61. Airflows and lamina airflows are particularly safe as a means for the adjacent persons meeting space 61. More aggressive sterilisation/extraction means may be used in the central meeting area 60 (e.g., fires and flames).

The efficiency of the sterilisation/extraction means used in the adjacent person meeting space 61 may be increased by other means which are not harmful to participants. These include a flow of negative ions drawn directly upwards by a positively charged electrostatic attractor above (as previously discussed). A beam of safe UV C may also be used as a sterilisation means in the configuration for the adjacent persons meeting space 61. Concentrated sunlight may be used to sterilize pathogens in the space 61. Hot air rising upwards may be used to destroy/remove pathogens in the space 61. Hot air at temperatures above 70 C may be used to destroy/remove pathogens in the space 61 without burning participants, because of the low heat capacity of air. This sort of embodiment provides more than one meeting space between persons.

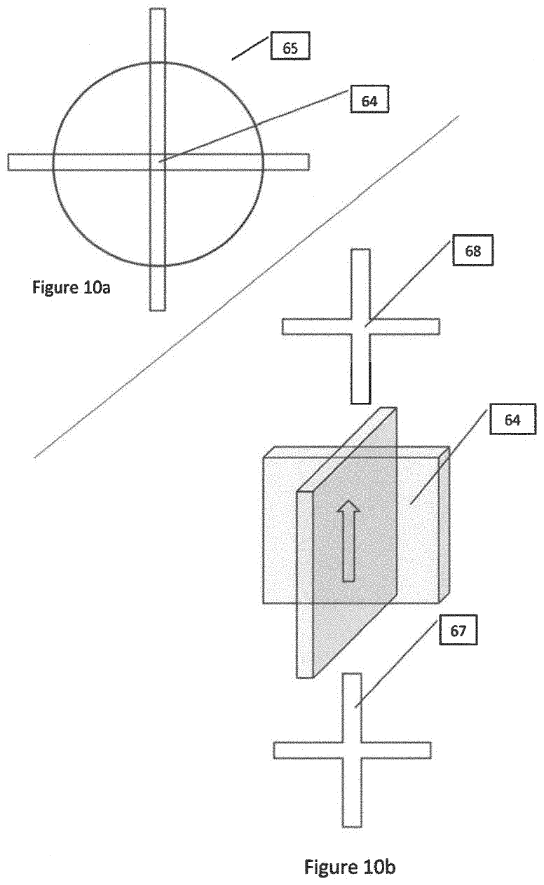

FIGS. 10a and 10b

FIGS. 10a and 10b are plan and side views of another possible embodiment of the invention. In this drawing, the means associated with the meeting spaces for persons using the inventive system are illustrated. In FIG. 10a, a sterilization/extraction means 64 is shown that is not harmful to participants in location 65. The sterilisation/extraction means 64 is shown from a side view in pseudo 3-D in FIG. 10b. Airflows and lamina airflows moving directly upwards as shown in FIG. 10b and are particularly safe as a barrier between participants. Location 65 is a safe sector for participants (a person location) as pathogens cannot pass through the ‘air-barrier’ 64.

The efficiency of the sterilisation/extraction means 64 may be increased by other means in addition to airflows, which are not harmful to participants. These include a flow of negative ions drawn directly upwards by a positively charged electrostatic attractor above. A beam of safe UV C may also be used as a sterilisation means 64 as configured in FIG. 10a. Concentrated sunlight may be used to destroy pathogens as part of means 64. Hot air rising upwards may be used to destroy pathogens as part of means 64. Hot air at temperatures above 70 Centigrade and even as high as 200 C may be used to destroy/remove pathogens as part of means 64 without burning participants, because of the low heat capacity of air.

FIG. 10b also shows another means 67 (in plan view) to create an additional upwards airflow/upwards laminar airflow in the configuration of means 64. Optionally, another means 68 positioned above the means 64 (also in plan view) may be used to provide means to extract air/or to create negative pressure causing an airflow/laminar airflow in the configuration of 64. Any known means to create the air flow or laminar air flow can be employed as part of the inventive system, either for the space between adjacent persons or the meeting space itself.

The means 67 may also create a flow of negative ions in the configuration of means 64 drawn directly upwards by a positively charged electrostatic attractor in means 68 above. Means 67 or 68 may produce safe UV C as a sterilisation means within the configuration of 64. Means 67 or 68 may create a beam of concentrated sunlight in the configuration of 64. Means 67 may produce hot air rising upwards in the configuration of 64. Optionally, means 68 may additionally provide extraction means so that the hot air rises directly upwards in the configuration of means 64.

This possible embodiment provides more than one meeting space and person locations outside the meeting spaces for persons using the meeting spaces, with no barriers between the meeting space(s) and the person locations such that persons in the person locations using the same meeting space(s) can communicate with each other through the meeting space(s). It is configured such that substantially all pathways within the system for the possible spread of pathogens between the person locations pass through at least one of the meeting spaces. Means are provided to make the meeting space(s) sterile or to substantially reduce the number of pathogens in the meeting spaces(s). It is configured to allow for persons in the person locations using the same meeting space(s) to hear each other through the meeting spaces(s) and for at least two persons using the same meeting space(s) to see each other through the meeting space(s).

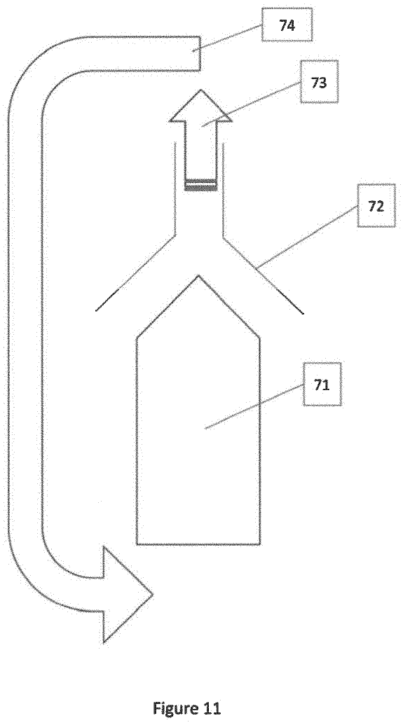

FIG. 11

FIG. 11 illustrates, in connection with the provision of one or more safe meeting spaces, an additional feature of some embodiments that utilizes the system to heat indoor or outdoor spaces and/or to remove pathogens from the space in which it is located. Additionally, it reduces the possible spread of pathogens between persons using different meeting spaces. In some embodiments, heated air 71 from the one or more meeting spaces is sufficiently hot to have destroyed all pathogens or to have destroyed substantially all pathogens. In some embodiments, the heated air 71 may be collected/extracted by collector/extractor 72 and the collected/extracted air 73 may be used to heat buildings and/or outdoor spaces.

The dual efficiency of using the same heat energy to create safe meeting spaces and also to heat buildings, is an important advantage of some embodiments the invention. In some embodiments, hot pathogen-free air is recycled back into the meeting space via a recycle stream 74, this increases the energy efficiency of the inventive system.

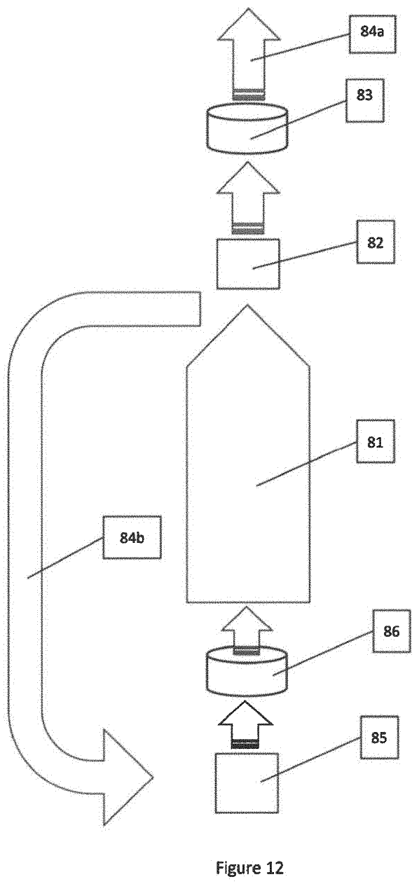

FIG. 12

Further to the provision of one or more safe meeting spaces, FIG. 12 illustrates additional embodiments that further utilize the inventive system to heat indoor or outdoor spaces and/or to remove pathogens from the space in which it is located. Additionally, this embodiment reduces the possible spread of pathogens between persons using different meeting spaces. In some embodiments, air, represented by the arrow 81, from the one or more meeting spaces, may contain pathogens. The air 81 from the one or more meeting spaces may be at room temperature or it may be heated but not heated sufficiently to destroy pathogens or to destroy substantially all pathogens. The air 81 may be filtered by filter 82 which uses known means to remove pathogens and/or the air 81 may be further heated by heater 83 to destroy all pathogens or to destroy substantially all pathogens. The clean air represented by the arrow 84a may be used to heat the space when the system is located.

In some embodiments, air from the meeting space(s) 81 may be recycled back into the meeting space(s) via stream 84b. In some embodiments, the air may be filtered by filter 85 and/or heated by heater 86 to remove/destroy pathogens before being returned to the meeting space(s). Recycling heated air back into the meeting space(s) can increase the energy efficiency of the system. In some embodiments warm clean air may be recycled into adjacent person meeting spaces, provided it is of sufficient temperature to destroy/inactivate pathogens but not sufficiently hot to harm persons.

The dual efficiency of using the same heat energy to create safe meeting spaces and also to heat buildings, is an important advantage of some embodiments the invention.

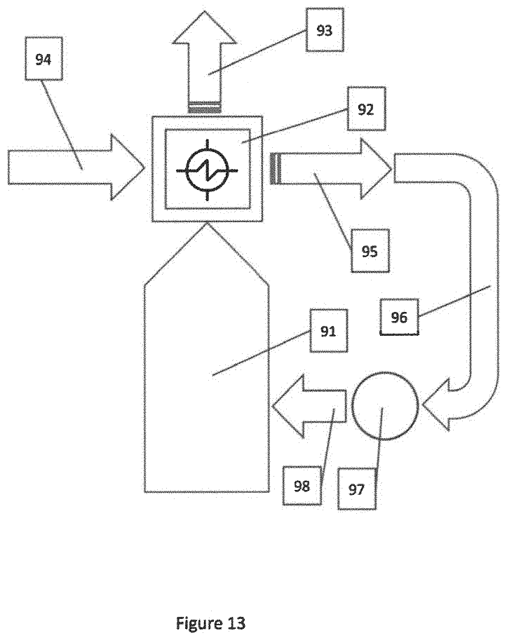

FIG. 13

Further to the provision of one or more safe meeting spaces, FIG. 13 shows another embodiment that further utilizes the inventive system to heat indoor or outdoor spaces and/or to remove pathogens from the space in which it is located. Additionally, it reduces the possible spread of pathogens between persons using different meeting spaces. In some embodiments, air designated by the arrow 91 from the one or more meeting spaces is heated but may contain pathogens. The heated air 91 may be passed through a heat exchanger or heat pump 92 to transfer heat to clean air, designated by arrow 94 or to transfer heat to water. After passing through the heat exchanger or heat pump 92 the air 91 which may contain pathogens is vented away as arrow 93. After receiving heat from the heat exchanger or heat pump 92 the heated clean air or heated water, designated by the arrow 95 may be used to heat buildings or outdoor spaces. In some embodiments where warm clean air 95 is used, it may be recycled into adjacent person meeting spaces, provided it is of sufficient temperature to destroy/inactivate pathogens but not sufficiently hot to harm persons.

In some embodiments, where arrow 95 constitutes hot clean air or warm clean air, the air may be supplied via recycle stream 96 to persons in person locations 97 around the meeting space. This feature increases the positive air pressure in person locations 97 in relation to the meeting space, which increases the flow of pathogens 98 from person locations 97 into the meeting space(s) (not shown). Increasing the flow of pathogens 98 from persons into the meeting space reduces the spread of pathogens between persons using different meeting spaces.

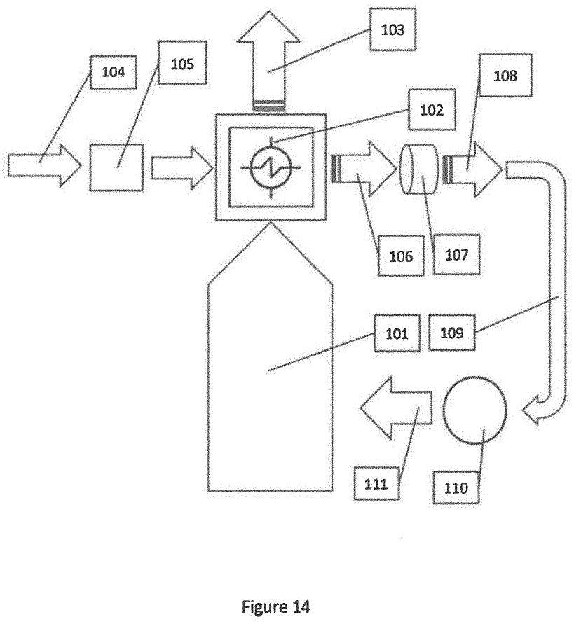

FIG. 14

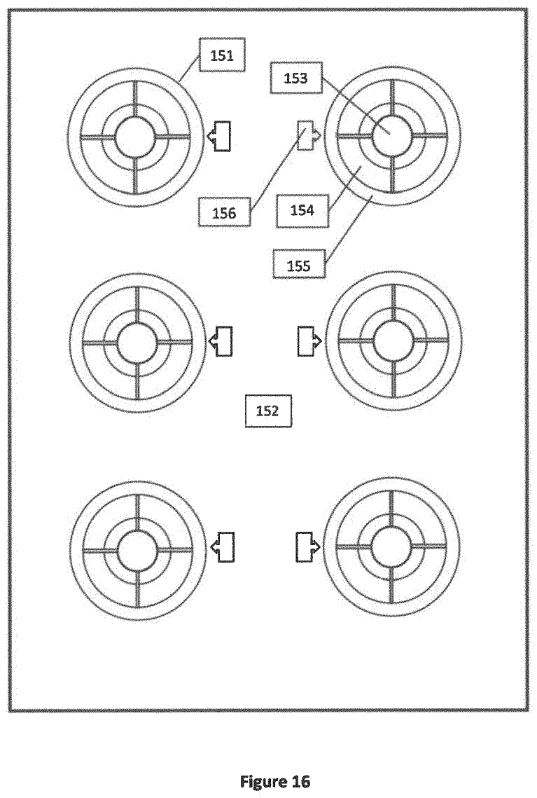

Further to the provision of one or more safe meeting spaces, FIG. 14 is another refinement of the invention and further utilizes the inventive system to heat indoor or outdoor spaces and/or to remove pathogens from the space in which it is located. Additionally, it reduces the possible spread of pathogens between persons using different meeting spaces. In some embodiments, air, which is designated as arrow 101 from the one or more meeting spaces of one or more systems is heated but may contain pathogens. The heated air 101 may be passed through a heat exchanger or heat pump 102 to transfer heat to air, designated by arrow 104 from the space (not shown) in which the invention is located. The space can be the space occupied by one system that uses a central meeting space, see FIG. 1 for example, and optionally adjacent person meeting spaces, see FIG. 8 for example, or more than one system, see FIG. 16 and its description below. After leaving the heat exchanger or heat pump 102, the unclean air is vented away as shown by arrow (103).

The air 104 from the space in which the invention is located may also contain pathogens. In some embodiments, the air 104 from the space in which the invention is located, may be passed through a filter 105 which uses known means to remove pathogens before being passed through the heat exchanger or heat pump 102. In some embodiments after leaving the heat exchanger or heat pump 102, the air, designated by arrow 106 is further heated by heater 107 to destroy pathogens. In some embodiments, the air 106 may be filtered to remove pathogens. The heated air 108 may be used to heat the space in which the invention is located and/or to provide clean pathogen-free air.

In some embodiments, hot clean air or warm clean air 108 may be supplied via recycle stream 109 to persons in person locations 110 around the meeting space. This feature increases the positive air pressure in person locations 110 in relation to the meeting space (not shown), which increases the flow of pathogens 111 from person locations into the meeting space(s). Increasing the flow of pathogens 111 from persons into the meeting space reduces the spread of pathogens between persons using different meeting spaces.

The sort of embodiment shown in FIG. 14 is effective at removing pathogens from the space in which it is located, as well as heating the space in which it is located, as well as providing one or more safe meeting spaces. In some embodiments hot clean air may be recycled into adjacent person meeting spaces, provided it is of sufficient temperature to destroy/inactivate pathogens but not sufficiently hot to harm persons.

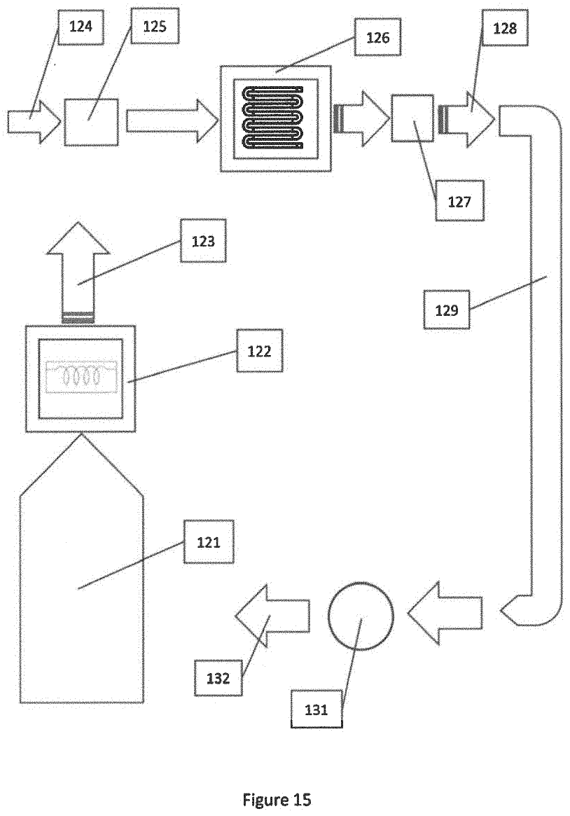

FIG. 15

Further to the provision of one or more safe meeting spaces, FIG. 15 illustrates another refinement that utilizes the inventive system to cool indoor spaces and/or to remove pathogens from the space in which it is located. Additionally, it reduces the possible spread of pathogens between persons using different meeting spaces. This sort of embodiment is useful in air-conditioned spaces.

Air, which is designated by arrow 121 and extracted from the one or more meeting spaces may be cooler than the external air temperature but it may contain pathogens. The air 121 may be used to cool the compressor coil of an air-conditioning unit/refrigeration unit 122 before being vented away as shown by arrow 123.

Air as shown by arrow 124 from the space in which the invention is located may also contain pathogens. In some embodiments, the air 124 from the space in which the invention is located, may be passed through a filter 125 which uses known means to remove pathogens before being passed through the evaporator coil of an air-conditioning unit/refrigeration unit 126. In some embodiments, the air is filtered by filter 127 to remove pathogens after leaving 126. The cool clean air, designated by arrow 128 may be used to cool the space in which the invention is located and/or to provide clean pathogen-free air.

In some embodiments, cool clean air 128) may be supplied via the recycle stream 129 to persons in person locations 131 around the meeting space (not shown). This feature increases the positive air pressure in person locations 131 in relation to the meeting space, which increases the flow of pathogens 132 from person locations into the meeting space(s). Increasing the flow of pathogens 132 from persons into the meeting space reduces the spread of pathogens between persons using different meeting spaces.

FIG. 16

FIG. 16 shows another embodiment of the invention comprising a system with multiple central meeting spaces. Means are provided to prevent pathogens passing between persons using different central meeting spaces.

One or a single system having the desired meeting spaces and means for preventing pathogens to pass between persons in the system is designated by the numeral 151, which is like the system shown with a central meeting space as found in FIG. 1, 8 or 9. The system of FIG. 16 shows six systems 151, all located in a space designated as 152.

Each of the systems 151 includes a central meeting space 153 and person locations 154 are shown surrounding the central meeting space 153. A zone 155 is provided that surrounds the person locations 154.

In some embodiments, the central meeting spaces 153 have extraction means and are at negative pressure in relation to the person locations 154. In some embodiments, the central meeting spaces 153 are at negative pressure in relation to the space in which they are located 152. In these embodiments the central meeting spaces act as wells for the extraction of pathogens from person locations and from the space in which they are located.

In some embodiments, means 156 are provided to supply clean (pathogen-free) air to the zone 155 surrounding person locations (154), increasing the positive pressure around person locations and increasing the flow of air into person locations, thereby increasing the flow of pathogens from the person locations into the central meeting spaces. This sort of embodiment also reduces the flow of pathogens from person locations into the space 152 where the systems 151 having the meeting space(s) are located. This sort of embodiment reduces the spread of pathogens between persons using different meeting spaces. The means 156 can be any means, such as those described above, that would supply clean (pathogen-free) air to a particular space or zone.

In some embodiments, clean (pathogen-free) air is supplied by the means 156 to person locations (154) or to the zone 155 surrounding person locations (154) or both, increasing the positive pressure in person locations in relation to the central meeting space, thereby increasing the flow of pathogens from the person locations into the central meeting space(s).

Clean airflows provided by the means 156 may be warm air as shown in FIGS. 13 and 14, or cool air as shown in FIG. 15.

In some embodiments, airborne negative ions are supplied to person locations 154 and/or the zone around person locations 155 and a positively charged electrostatic attractor is provided in the central meeting space 153 to increase the flow of pathogens into the central meeting space.

### FURTHER DETAILED DESCRIPTION AND ADDITIONAL FEATURES

When discussion topics are displayed as part of the sterile and pathogen free meeting place, the means for creating the safe space by sterilization means/techniques can also be used to either display the discussion topics for the participants or enhance such displays provided by other means. For example, discussion topics can be displayed as part of a fire, UV beams can be used to illuminate fluorescent displays, images can be projected into smoke, etc.

Additional features of the system include the use of sound reflectors.

In some embodiments, the invention provides a central meeting space through which face-to-face conversation/discussion occurs. Sound waves from participants may pass through the meeting space but pathogens may not. This means that participants can have natural conversations through air, without being impeded by a physical barrier such as a glass screen. In the method of the invention, pathogens may not pass from one participant to another through the central meeting space.

This application includes both the apparatus and the method described herein. It also includes the use of the invention. The invention may be used to accommodate safe face-to-face meetings between participants, while avoiding/reducing the risk of spread of communicable diseases, such as Covid-19. This invention includes the use of the invention by businesses and other organizations to provide safe face-to-face meetings between participants and safe in-person activities.

The invention may be used for a wide variety of face-to-face events and/or in-person activities, these include but are not limited to: Networking events, business meetings, conferences, conventions, discussion forums and educational and training activities. Further examples of some of the commercial uses of the invention are listed elsewhere below.

Additionally, the apparatus and method of the invention may be used to make safe other in-person, group activities or public places including and not limited to: Restaurants, areas where customers eat together, use in bars, pubs and drinking establishments, entertainment and performance spaces, museum and galleries, shops and retail spaces.

Additionally, this invention may be used as a new refinement to U.S. Pat. No. 7,111,244 and United States Pre-Grant Publication No. 2020/0334765, each to Norris and incorporated by reference in their entirety herein (hereinafter the Norris patent and the Norris publication, respectively), to allow safe face-to-face networking and group discussion between participants. It may be used independently to accommodate safe face-to-face meetings between participants, while avoiding/reducing the risk of spread of communicable diseases, such as Covid-19.

In some embodiments of the invention, an up-flow is created in the meeting space taking pathogens up and away from participants. In some embodiments. Fresh clean airflows may be provided for participants, and air from the meeting area may be vented away, and/or pumped away which further facilitates the clean airflow to participants.

In some embodiments, participants are typically two metres apart, in line with current social distancing guidelines. Communication takes place through the safe meeting space. In some embodiments, participants who are much closer together, for example those sitting adjacent may be separated by partitions or walls. These partitions may be transparent so participants may see each other. These partitions ensure that there is no airflow and no spread of pathogens between participants. Face-to-face communication only takes place through the sterile meeting space. In some embodiments, participants are inside separate chambers connected only via the safe meeting space.

An additional feature of some embodiments is the use of the means to create a pleasing visual focus for discussion and/or to create a stimulating environment for conversation. A further feature of some embodiments is the use of the means to displaying subjects for discussion in the meeting space(s).

In some refinements to the invention, discussion topics may be displayed in the meeting space or nearby. These are subjects for discussion by the participants. In some embodiments the sterilisation means and/or the means for removing pathogens are used to display discussion topics. This dual purpose efficiency is a further advantage. This means that participants may choose to join a particular discussion group that interests them as indicated by the discussion topic on display. New discussion groups can be created by displaying new topics, in accordance with the Norris patent and publication.

In some embodiments, a central meeting space may be sterilised/cleaned by any one or more of the following means, and/or pathogens are removed from that space:
1. A fire or heating device is provided in the meeting space, which raises the temperature of the air in that space to destroy pathogens. It also creates a convective updraft thereby removing pathogens from the meeting space and away from participants. Additionally, this draws fresh clean air in towards participants.
2. Sunlight or concentrated sunlight may be used to sterilise the meeting space and/or to power a heating device to create a convective updraft. Solar energy may also be used to power any of the other sterilisation/cleaning means described here.
3. A UV source may be used to sterilise the central meeting space.
4. An electrostatic attractor placed above the sterile meeting space and/or above the participants to draw airborne pathogens up and away from participants.
5. Smoke, fumigants, mists, vapours and gases may be used to sterilise the meeting space. These may be produced by fires as in (1) above, or produced by solar heat energy or devices powered by electrical energy and/or solar energy. The smoke, fumigants, mists and vapours may contain agents that destroy or inactivate pathogens.

The invention may be used indoors or outdoors, to facilitate safe face-to-face conversation between participants. The sterilization means utilised here—such as fires and are known to enhance conversation when people sit around them. Similarly, glowing mists and aromatic vapours provide a pleasant focus for discussion. UV light and electrostatic fields can create exciting visual effects, particularly at night. This further enhances the user experience and is a further advantage of the invention. Solar powered embodiments of the invention provide shade and a cooling inflow of clean fresh air. These solar embodiments of the invention are sustainable and carbon neutral.

An advantage of using the sterilisation means to create a pleasing visual effect is that it creates a focus for discussion and/or conversation. A further advantage of the visual effect is that it reassures persons that the sterilisation means are fully operational, particularly when those means are invisible.

Partitions (walls) may be placed between participants who are adjacent to each other. These partitions ensure that there is no airflow and no spread of pathogens between participants. These partitions may be transparent so participants may see each other. Face-to-face communication only takes place through the sterile meeting space.

In some embodiments, one or more meeting space(s) may be sterilised and/or cleaned, by one or by combinations of the following means:
1. A fire or heating device in the meeting space which raises the temperature of the air in the space. The fire or heater, also creates an updraft taking pathogens up and away from the participants. Additionally, fresh clean air is drawn into the area where participants are located. IR heat radiation may be reflected back into the meeting space to increase its capacity to destroy pathogens.
2. Sunlight or concentrated sunlight may be used to sterilize the meeting space. Solar powered embodiments of the invention are particularly useful for outdoor applications such as on beaches and in places of natural beauty. In some embodiments solar-tracking means are also provided. the sunlight/concentrated sunlight may strike an absorbent target (ideally a black body) which acts as a heater, creating a thermal updraft, similar to (1) above. The green-house effect may be utilised to increase the temperature of the target, by converting more sunlight to IR. The target absorbs sunlight and emits heat by radiation and convection. It may be concave/parabolic and/or contain heat emitters such as fins. The target may absorb sunlight in the day and by means of a heat storage device, it may re-emit heat energy at night. Sunlight may be concentrated into the meeting space by mirrors, or lenses and Fresnel lenses, all of which are well known. The UV content of sunlight may be concentrated using a concave aluminium reflector or similar material. Aluminium is also effective at reflecting and concentrating IR to heat a target.
   Aluminium's relatively low specific heat capacity means that it is will absorb less heat energy and it is a good choice for light-weight construction. Lenses (and Fresnel lenses) to concentrate solar UV may be made from suitable refractive materials such fused silica. Likewise, lenses (and Fresnel lenses) to contrate solar IR may be made from suitable refractive materials such as germanium or calcium fluoride. Composite lenses (or composite Fresnel lenses) may concentrate both IR and UV separately with different focal points for each. IR may be used to heat an absorbent target, while UV may be reflected back into a sterilisation beam. A lens may focus and convert most of the sunlight to IR to heat an absorbent target. The visible component of sunlight may be used to illuminate display devices as described below.
   Some embodiments of the invention are energy efficient, carbon neutral and sustainable. Some embodiments utilise solar energy. Some solar embodiments of the invention separate sunlight into UV, IR and visible light to provide multiple and/or complementary means to destroy, inactivate and/or remove pathogens.
   In some embodiments of the invention, different focal zones are created for solar UV, visible and IR radiation, typically but not exclusively by refraction. The focal zones may be static if solar tracking means are provided—or a larger zone allowing for the movement of the sun. In some embodiments, the focal zone for IR will be below the zone for visible light, which is below the UV zone. In these embodiments the IR component may be used to heat an absorbent target as described above, and/or to vaporise an antipathogenic agent. The UV component may be reflected back upwards to increase the intensity of the UV sterilisation beam. In some embodiments the reflector for UV is transparent to IR and in some embodiments, it is also transparent to visible light. In some embodiments, solar UV is focused into a hollow tube-like beam around the perimeter of the central meeting space. This ensures that pathogens entering the central meeting space always pass through the UV beam. Spreading the solar UV beam across the entire central meeting space would reduce its intensity. The visible light component may be to illuminate a display screen as outlined in sections below. In some embodiments all sunlight is focused into a hollow tube-like beam around the perimeter of the central meeting space. This ensures that pathogens entering the central meeting space always pass through the solar beam.
   Solar energy may be captured using electronic solar panels. It may be stored for use at night-time to power sterilisation means. Solar energy may power a heater to provide a thermal updraft to carry pathogens up and away from participants. It may also power an electrostatic attractor as outlined in (4) or a UV device at night as in (3) below. Solar energy may also be used to ionise particles described in 5 below, differently to an attractor. Solar energy may be used to vaporize antipathogenic agents, such as alcohol.
3. A UV source may be used to sterilize the central meeting space. Additionally, UV may be utilised to generate ozone in the central meeting space, particularly in the presence of an electrostatic field as in (4) below. Ozone is known to destroy pathogens, when differently charged to them, it binds to them. In some embodiments UV is focused into a hollow tube-like beam around the perimeter of the central meeting space. This ensures that pathogens entering the central meeting space always pass through the UV beam. Spreading the UV beam across the entire meeting space would reduce its intensity.
4. An electrostatic attractor above the fire/heating device and/or above the participants may be used to draw airborne pathogens up and away from participants. This technology is well known for removing soot particles from fires, but may also be utilised to drawn airborne pathogens up and away from participants. Pathogens in the meeting space may be ionised differently to the attractor, e.g., with the opposite charge, as in an electrostatic filter. Solar energy may be used to power the electrostatic attractor as in 2 above. Static electricity may be extracted from the environment using known technologies. RF antennae may also collect electrical energy. Solar energy may be used to create the static electricity for this process, for example by the photoelectric effect.
5. Smoke, fumigants, mists, vapours and gases may be used to sterilise the meeting space. The smoke, fumigants, mists, vapours and gases may contain agents that destroy or inactivate pathogens. These may be produced by fires as in (1) above, other known means such as ultrasonic vaporiser or an evaporator may also be utilised. The anti-pathogenic smoke, fumigants, mists, vapours and gases may also be carried upwards into a thermal updraft by a heating device or fan. Optionally additionally, an electrostatic attractor, as in (4) may be used to draw smoke, fumigants, mists and vapours into the central meeting space, then up and away from participants (unless like alcohol vapour they may enjoy it)! Safe Aromatic oils may be added to give a pleasant aroma. Solar energy may also be used to ionise the particles described here.
6. Combining methods 4 and 5, an ionising device may be used to vaporise an antipathogenic agent, or simply to vaporise an agent which binds to the airborne pathogens. The particles comprising the smoke, fumigants, mists and vapours may be given the opposite electrical charge to an electrostatic attractor (typically above) or it is given a different charge. Additionally, giving the particles of smoke, fumigants, mists and vapours an electrostatic charge, different to the airborne pathogens, will increase their binding and attraction to those pathogens. Pathogens bound to the particles of smoke, fumigants, mists and vapours will be drawn towards the opposite charged electrostatic attractor. This method is novel and different to standard electrostatic precipitators and de-ionizers. It may also be used for other applications—which involve removing airborne pathogens from a space such as a room.

The use of multiple and/or complementary means in some embodiments of the invention is an important advantage, as it increases the efficiency of the sterilisation/extraction process. For example, some solar powered embodiments of the invention use both IR and UV radiation as well as convective airflows to maximise the sterilisation/extraction process.

Partitions (walls) may be placed between participants sitting adjacent to each other. These partitions may be transparent so participants may see each other. These partitions ensure that there is no airflow and no spread of pathogens between participants. Face-to-face communication only takes place through the sterile meeting space. Optionally additionally, walls or similar barriers may be placed around the invention, particularly outdoors, to mitigate winds etc that may spread pathogens beyond the central meeting space.

Fresh clean airflows may be provided for participants, and air from the meeting area may be vented away, and/or pumped away which further facilitates the clean airflow to participants. For indoor embodiments of the invention, air from the meeting space may be made to flow outside of the building.

Sound reflectors may be placed behind participants, to focus sound waves into the sterile meeting space. These sound reflectors may be concave, the participant is in the focal area directing sound waves into the meeting space. Sound waves coming from the meeting space will be directed towards participants. The use of these sound reflectors could increase the safe social distancing between users. Further reducing the risk of infection. Sound reflectors may therefore be used without sterilisation means. This provides the following benefits:
1. Sound from participants is directed into the sterile meeting area.
2. The use of these sound reflectors may increase the safe social distancing between users. Further reducing the risk of infection. Sound reflectors may therefore be used without sterilisation means.
3. Sound coming from the central area is directed towards participants
4. Sound from the discussion/conversation does not affect other groups of participants using another embodiment of the invention nearby.
5. This type of embodiment is particularly suitable for sterilisation/extraction means that attenuate and dampen sound

Sound reflectors may also be used as a further inventive refinement to the Norris patent and publication.

Displaying Discussion Topics (Subjects for Discussion Between Participants)

Further to the Norris patent and publication, discussion topics may be displayed at locations where participants enter into discussion with each other.

The sterilisation/cleaning means may be utilised to display subjects for discussion. The efficient dual use of the sterilisation/cleaning means, to enable safe face-to-face conversation, and also to display discussion topics is a further inventive refinement to both inventions. In some preferred embodiments the discussion topic appears in the air.

The sterilisation/extraction means may be utilised to display subjects for discussion, as follows:
1) Fires and flames: The Norris publication describes how discussion topics may be displayed in fires, while users sitting around those fires may engage in networked face-to-face discussion. Additionally, that fire may be used to create a safe sterile meeting space between those participants as outlined here. The Norris publication states “one or more discussion signs are associated with an actual fire . . . and optionally the discussion signs form part of the actual fire . . . , or the discussion topic appears within the fire . . . as an area of greater brightness, darkness or in contrasting colors”. Scanning lasers or projectors may create images or words in fires, displaying discussion topics by exciting particles in the fire to emit light, or to reflect light as in Tyndall effect. Alternatively, a standard electronic display screen (such as LED or QLED) may display discussion topics nearby in contrasting colours.
2) Discussion topics can also be projected into smoke, mists and vapours which contain antipathogenic agents. According to the Norris publication: “the discussion sign is projected into smoke, fire, mist, steam or vapor at discussion locations or into any medium containing particles capable of creating an image through the Tyndall effect”. Scanning lasers or projectors may create images or words in smoke, mists and vapours displaying discussion topics, by exciting particles in the smoke, mists or vapours to emit light or to reflect light by the Tyndall effect. Alternatively, a standard electronic display screen (such as LED, OLED or QLED) may display discussion topics in contrasting colours.
3) UV light can be used to illuminate fluorescent LCD screens to display a discussion topic in visible light. A UV based LCD projector can display discussion topics on a fluorescent screen as visible light. Discussion topics can be projected as UV light into a vapour, smoke or mist which is fluorescent. Additionally, the fluorescent vapour, smoke or mist may be antipathogenic or contain antipathogenic agents as in 2 above. Alternatively, a standard electronic display screen (such as LED, OLED or QLED) may display discussion topics in contrasting colours.
4) For embodiments of the invention that capture sunlight; some of that sunlight may be used to illuminate an LCD screen which displays discussion topics. Additionally, the sunlight may be reflected by the LCD screen and/or optionally focused onto a screen. Alternatively, sunlight may pass through a transparent LCD screen and focused onto a screen to display discussion topics in the manner of an LCD projector. Visible sunlight may be concentrated in a different place to IR and UV, when it may illuminate a display unit. Alternatively, a standard electronic display screen (such as LED, OLED or QLED) may display discussion topics in contrasting shades to the sunlight e.g., black.
5) Alternatively, electrical energy from solar power may be stored and/or used to operate a display device. Particularly at night when sunlight is not available.
6) Electrostatic fields may generate light for example by corona discharge, this would enable discussion topics to be displayed in the sterile meeting space—or nearby. Gases, particles and other molecules emit light in an electric field. A scanning laser or a projector could display discussion topics in a contrasting colour. Inert gases such as Argon and Neon emit light in an electrostatic field. They could be encased in a separate transparent chamber (typically at low pressure) inside the meeting space. A scanning laser or projector could display discussion topics in a contrasting colour. Alternatively, these fluorescent gases could illuminate an LCD display, or light from them could pass through a transparent LCD display screen. Alternatively, a standard electronic display screen (such as LED, OLED or QLED) may display discussion topics in contrasting colours.
7) Ozone in the meeting space can cause other substances to glow. This effect can be utilised to display discussion topics in the meeting space. A scanning laser or projector could display discussion topics in a contrasting colour. Alternatively, a standard electronic display screen (such as LED, OLED or QLED) may display discussion topics in contrasting colours.

Virtually any known antipathogen capable of killing bacteria, viruses, and the like can be used in the inventive system that can be dispersed in the central meeting space, either on its own as attached to a carrier for dispersion. Chlorine-based antipathogens, e.g., chlorine dioxide or antimicrobials/antivirals, e.g., hydrogen peroxide, and ozone, that can be dispersed, are just examples. Also, any known carriers capable of carrying an antipathogen for dispersion in the central meeting space can be used as part of the inventive method and system.

This application includes both the apparatus and the method described herein. It also includes the commercial use of the invention by businesses and other organisations. The invention may be used for a wide variety of face-to-face events and/or in-person activities, these include but are not limited to: Networking events, business meetings, consultation meetings, focus groups, conferences, conventions, discussion forums, discussion groups and educational activities, social events involving conversation or discussion. Additionally, the invention may be used to facilitate safe social interaction in bars, cafes and nightclubs. The invention provides significant commercial advantage to certain businesses or organisations by providing a new revenue stream and/or by enhancing the existing business and/or by driving new customers to that business, typically by providing safe face-to-face meetings and safe face-to-face social interaction.

The invention may be used by events companies, event's organizers, entertainment companies, networking companies, social networking companies or business networking companies. Similarly, universities, educational organizations, training providers, professional associations, personal or business development organisations, or professional development organisations could benefit from the use of the invention to offer events and activities that are directly related to their core business. Social media companies and social networking companies could gain significant commercial advantage by offering face-to-face communication alongside online communication.

Some preferred embodiments of the invention are configured so that all pathways for the possible spread of pathogens between persons pass through sterilization means/pathogen extraction means. In some preferred embodiments of the invention persons share the same acoustic space and can hear each other. In some preferred embodiments of the invention persons can see each other.

In another embodiment, the meeting space could be configured to allow one or more musicians to occupy at least a part of the space. The configuration would be such that the participants or persons surrounding the meeting space would be able to see and hear the musicians but still be protected by portions of the meeting place still arranged between the musicians and the participants or persons surrounding the meeting space.

The invention may be used by a plurality of musicians so that sound may pass freely between them but pathogens may not. This provides the advantage that musicians are able to make music within the same acoustic space but are protected from the spread of communicable diseases.

In yet a further embodiment, instead of using partitions to separate persons who are adjacent each other, means similar or the same as that used for the meeting space to create a barrier which is not so physical but still prevents adjacent persons from being exposed to pathogens from each other when using the meeting space. In this way, the persons adjacent to each other and also using the meeting space are not separated by a physical barrier but are still protected.

In one embodiment, the means used to create the sterile or substantially pathogen free space between adjacent persons is linked to the means used to create the same for the meeting space. In this way, the same means that creates the environment in the meeting space can be used to establish a barrier or sterile or substantially pathogen free space between adjacent persons, thus providing a more efficient system and method as compared to one that used different means to create a safe space between adjacent persons. It should be understood that when using the means for creating the safe space for the meeting place for the space between adjacent persons, any means that would present a potential danger to the adjacent persons would not be included. For example, while fire is exemplified to sterilize the meeting place, the close proximity between adjacent participants would weigh against the use of fire. Similarly, instances where intense radiant heat is used for sterilization purposes in the meeting place, such radiant heat would not be appropriate between adjacent persons as it may present a risk of burning. Further yet, if any airborne materials or carriers that would be used for sterilizing the meeting space would be hazardous to adjacent persons due to the reduced size of the space between adjacent persons as compared to the size of the meeting space, such materials or carriers would not be appropriate to create a safe space between adjacent persons.

However, the system and method can also be configured such that the means for creating the safe space between the adjacent persons can be different from that used in conjunction with the meeting space but still provide the safe barrier and space so as to prevent pathogens from traveling between adjacent participants. For example, where concentrated sunlight is used in the meeting space, air flows and laminar air flows could be used to create a barrier to pathogens passing between adjacent persons.

In any event, the means replacing the partitions in the space between adjacent person is for creating an environment in the space that is sterile or substantially pathogen free such that the adjacent persons can be next to each other without any physical barrier between the persons and without worry of becoming ill as a result of the being adjacent to each other, the means for creating either: directly killing the pathogens that may be in the meeting space; or removing pathogens from the meeting space, or a combination of the two.

In some embodiments, replacing the partitions between adjacent persons with an ‘adjacent person meeting space’ offers many advantages. Firstly, the invention becomes less obtrusive, it feels like an open natural space without barriers between persons, thereby improving the user experience. Secondly, adjacent persons can talk to each other naturally and freely, improving the quality of communication.

One example of creating the barrier or space that is sterile or substantially pathogen free for adjacent persons is the use of air flow, both laminar and convective. The efficiency of the sterilisation/extraction barrier between adjacent participants may be increased by other means in addition to airflows, which are not harmful to participants. These include a flow of negative ions drawn directly upwards by a positively charged electrostatic attractor above. A beam of safe UV C may also be used as to sterilize the air. Concentrated sunlight may be used to destroy pathogens passing through the ‘air barrier’ between adjacent participants. Hot air rising upwards may be used to destroy pathogens between adjacent participants. Warm air at temperatures above 70 Centigrade and even above 100 Centigrade may be used to destroy pathogens, without burning participants, because of the low heat capacity of air. As noted above, it should be understood that if the air barrier is supplemented with other means for pathogen removal or suppression, the means can be those described for the meeting space provided that they do not present any danger to the adjacent participants.

More specific examples of the means for creating a sterile environment between adjacent persons includes:

more of:
i) devices using elevated temperatures, with an optional exhaust;
ii) lenses and or mirrors to created concentrated sunlight, and to generate UV and/or IR radiation using sunlight;
iii) safe ultraviolet light (UV C) for pathogen destruction;
iv) one or more electrostatic attractors to draw pathogens away; and
v) the use of one or more airborne materials in the meeting space, the airborne materials for directly sterilizing the meeting space or acting as a carrier for another sterilant or anti-pathogen, and means to move the airborne materials through the meeting space, the airborne materials safe for being in proximity to adjacent participants.
vi) means for generating airborne negative ions within the space between adjacent persons, which may bind to airborne pathogens to inactivate them. Optionally and preferably a positive electrostatic attractor to draw those negative ions up through the space between adjacent persons, as well as for removing pathogens, including those pathogens that have become negatively charged by binding to the negative ions.

Just like for the embodiment that provides means for creating a sterile or substantially pathogen free space for the meeting space and indicia of discussion topics for the face to face discussion, the space between adjacent participants can also employ such indicia as well.

Further examples of the commercial use of the invention are listed below.
1. Advertising or promotional or sales events and/or advertising or promotional or sales events that involve face-to-face conversation
2. Meetings for the provision of business services or customer services;
3. Events for the provision of financial services;
4. Focus groups, and optionally creating focus groups for market research or for product development or for political organizations;
5. Political events;
6. Cultural events;
7. Conventions;
8. book fairs;
9. trade shows;
10. community events;
11. civic events;
12. personal development events;
13. professional development events;
14. improving communication within companies or organizations;
15. improving communication between companies or between organizations;
16. group therapy or other therapeutic use based on face-to-face conversation;
17. medical or therapeutic use by providing social interaction;
18. medical or therapeutic use by providing social connection;
19. medical or therapeutic use by connecting patient groups with related medical conditions;
20. medical or therapeutic use by sharing experiences of medical conditions,
21. educational use to teach or learn communication skills or networking skills or social skills;
22. educational use that involves teaching or learning by face-to-face discussion
23. recruitment events;
24. matching job applicants to employers;
25. networking between job applicants and employers;
26. mediation events, mediation between groups or individuals;
27. conflict resolution events;
28. dating events,
29. networking events arising from conferences or conventions; and networking events arising from conferences or conventions involving the creation of breakout groups,
30. business networking,
31. social networking,
32. professional career networking,
33. expert networking,
34. expert networking in areas including medicine and related professions, science, biotechnology, pharmaceuticals, technology, engineering, computing, electronics, digital technology.

The invention can also be characterized in one embodiment by the following:

1. A system that facilitates face to face discussions between two or more persons via a meeting space without the need for a barrier between the two persons comprising:
a) means to separate or space apart the persons and establish a meeting space there between;
b) means for creating an environment in the meeting space that is sterile or substantially pathogen free such that the persons can have a face to face discussion using the meeting place without any physical barrier between the persons and without worry of becoming ill as a result of the face to face discussion, the means for creating either: directly killing the pathogens that may be in the meeting space; or removing pathogens from the meeting space, or a combination of the two;
c) optionally means to move fresh air through the meeting space; and
d) optionally one of:
   partitions positioned in a space between adjacent persons that are not face to face; or
means for creating an environment in the space between adjacent persons that is sterile or substantially pathogen free such that the adjacent persons can be next to each other without any physical barrier between the persons and without worry of becoming ill as a result of the being adjacent to each other, the means for creating either: directly killing the pathogens that may be in the meeting space; or removing pathogens from the meeting space, or a combination of the two, and not presenting a danger to either of the adjacent persons.

2. The system of 1, wherein the creating means for the meeting place comprise one or more of:
i) devices employing fire and/or elevated temperatures, with an optional exhaust;
ii) means to heat surfaces, generate infrared radiation from a surface for sterilization, use lenses to created concentrated sunlight, and to generate UV and/or IR radiation using sunlight;
iii) ultraviolet light for pathogen destruction;
iv) one or more electrostatic attractors to draw pathogens away; and
v) the use of one or more airborne materials in the meeting space, the airborne materials for directly sterilizing the meeting space or acting as a carrier for another sterilant or anti-pathogen, and means to move the airborne materials through the meeting space.

3. The system of 1 or 2, wherein the creating means for the meeting place also provides an indicia of discussion topics for the face to face discussion or enhance an existing discussion topic associated with the meeting space.

4. The system of 1 to 3, further comprising the use of sound reflectors to enhance communication between the persons.

5. The system of any of 1 to 4, wherein the means for creating an environment in the space between adjacent persons further comprises:
i) devices using elevated temperatures, with an optional exhaust;
ii) lenses or mirrors to created concentrated sunlight, and to generate UV and/or IR radiation using sunlight;
iii) safe ultraviolet light (UV-C) for pathogen destruction;
iv) one or more electrostatic attractors to draw pathogens away;
v) the use of one or more airborne materials in the meeting space, the airborne materials for directly sterilizing the meeting space or acting as a carrier for another sterilant or anti-pathogen, and means to move the airborne materials through the meeting space, the airborne materials safe for being in proximity to adjacent participants; and
vi) means for generating airborne negative ions within the space between adjacent persons, which may bind to airborne pathogens to inactivate them. Optionally and preferably a positive electrostatic attractor to draw those negative ions up through the space between adjacent persons, as well as for removing pathogens, including those pathogens that have become negatively charged by binding to the negative ions.

6. The system of 1 to 5, wherein the creating means for the safe space between adjacent person also provides an indicia of discussion topics for the face to face discussion or enhance an existing discussion topic associated with the meeting space.

7. A method of using the system of any one of 1 to 6, wherein the meeting space is used for one or more of the following:
1. Advertising or promotional or sales events and/or advertising or promotional or sales events that involve face-to-face conversation
2. Meetings for the provision of business services or customer services;
3. Events for the provision of financial services;
4. Focus groups, and optionally creating focus groups for market research or for product development or for political organizations;
5. Political events;
6. Cultural events;
7. Conventions;
8. book fairs;
9. trade shows;
10. community events;
11. civic events;
12. personal development events;
13. professional development events;
14. improving communication within companies or organizations;
15. improving communication between companies or between organizations;
16. group therapy or other therapeutic use based on face-to-face conversation;
17. medical or therapeutic use by providing social interaction;
18. medical or therapeutic use by providing social connection;
19. medical or therapeutic use by connecting patient groups with related medical conditions;
20. medical or therapeutic use by sharing experiences of medical conditions,
21. educational use to teach or learn communication skills or networking skills or social skills;
22. educational use that involves teaching or learning by face-to-face discussion
23. recruitment events;
24. matching job applicants to employers;
25. networking between job applicants and employers;
26. mediation events, mediation between groups or individuals;
27. conflict resolution events;
28. dating events,
29. networking events arising from conferences or conventions; and networking events arising from conferences or conventions involving the creation of breakout groups,
30. business networking,
31. social networking,
32. professional career networking,
33. expert networking,
34. expert networking in areas including medicine and related professions, science, biotechnology, pharmaceuticals, technology, engineering, computing, electronics, digital technology, and
35. a musical performance.

In some embodiments the invention comprises apparatus that provides at least one meeting space that is pathogen-free or substantially pathogen free for two or more persons without the need for a barrier between the two or more persons in which:

a) the apparatus is configured so that all pathways for the possible spread of pathogens between persons pass through the at least one meeting space;

b) means are provided to at least substantially reduce the number of pathogens in the at least one meeting space; and

c) the apparatus is configured so that sound may pass through the at least one meeting space thereby enabling persons to hear each other; and

d) the apparatus is configured so that at least two or more persons may see each other.

In some embodiments, the invention comprises apparatus that provides a meeting space that is pathogen-free or substantially pathogen-free for two or more persons without the need for a barrier between the two or more persons comprising:
a) means to separate or space apart the persons and establish the meeting space there between;
b) means for making the environment in the meeting space sterile or substantially pathogen free such that the persons can have a face-to-face discussion using the meeting space without any physical barrier between the persons and without worry of becoming ill as a result of the face-to-face discussion, the means at least substantially reducing the number of pathogens in the meeting space; and
c) providing partitions positioned in the space between adjacent persons using the meeting space that are not fully separated by the meeting space so that pathogens may not pass between the adjacent persons.

In some embodiments, the invention comprises a method to provide at least one meeting space that is pathogen-free or substantially pathogen-free for two or more persons without the need for a barrier between the two or more persons comprising:

a) providing apparatus that is configured so that all pathways for the possible spread of pathogens between persons pass through the at least one meeting space;

b) providing means to at least substantially reduce the number of pathogens in the at least one meeting space; and

c) configuring the apparatus so that sound may pass through the at least one meeting space thereby enabling persons to hear each other; and

d) configuring the apparatus so that at least two or more persons may see each other.

In some embodiments, the invention comprises a method to provide a meeting space that is pathogen-free or substantially pathogen-free for two or more persons without the need for a barrier between the two or more persons comprising:
a) providing means to separate or space apart the persons and establish the meeting space there between;
b) providing means for making the environment in the meeting space sterile or substantially pathogen free such that the persons can have a face-to-face discussion using the meeting space without any physical barrier between the persons and without worry of becoming ill as a result of the face-to-face discussion, the means at least substantially reducing the number of pathogens in the meeting space; and
c) providing partitions positioned in the space between adjacent persons using the meeting space that are not fully separated by the meeting space so that pathogens may not pass between the adjacent persons.

In some embodiments the invention provides means to at least substantially reduce the number of pathogens and further comprises means to substantially destroy and/or inactivate pathogens in the at least one meeting space so that the pathogens cease to be infectious.

In some embodiments, the means to at least substantially reduce the number of pathogens further comprises substantially removing the pathogens from the at least one meeting space.

In some embodiments of the invention, the means further comprises one or more of the following:
a) at least one heat source and/or heated air produced by a heat source;
b) ultraviolet and/or infrared and/or solar radiation;
c) at least one electrostatic attractor and/or at least one kind of airborne ion;
d) at least one anti-pathogenic agent; and
e) at least one airflow.

In some embodiments of the invention the means for substantially and/or completely destroying and/or inactivating and/or removing pathogens is one or more of the following:

a. fire and/or flames and/or elevated temperatures;

b. fire and/or flames and/or elevated temperatures with an exhaust;

c. convective up-flow of air to remove pathogens from the meeting space;

d. hot air in the meeting space to destroy or inactivate pathogens therein;

e. hot air of sufficient temperature to destroy or inactivate airborne pathogens without burning persons;

f. hot surfaces that generate infrared radiation for sterilization;

g. sunlight concentrated in the meeting space;

h. UV and/or IR radiation from sunlight;

i. UV and/or IR from sunlight concentrated in the meeting space;

j. ultraviolet light for pathogen destruction/deactivation;

k. one or more electrostatic attractors to draw pathogens away from the meeting space

l. one or more electrostatic attractors to draw pathogens away from persons using the meeting space;

m. one or more electrostatic attractors to attract pathogens where the attractor is heated to destroy pathogens on contact;

n. heated and/or vaporized antipathogenic agents in the meeting space;

o. antipathogenic agents made airborne in the meeting space by ultrasound;

p. safe ultraviolet light (UV-C) for pathogen destruction;

q. airborne ions in the meeting space that bind to pathogens;

r. airborne negative ions in the meeting space that bind to pathogens;

s. airborne ions in the meeting space and one or more differently charged electrostatic attractor to draw the airborne ions through the meeting space;

t. airborne negative ions in the meeting space and one or more positively charged electrostatic attractor to draw the airborne ions through the meeting space;

u. ionized airborne antipathogenic agents in the meeting space;

v. ionized airborne antipathogenic agents in the meeting space and one or more differently charged electrostatic attractor to draw the airborne ions through the meeting space;

w. ionized airborne antipathogenic agents in the meeting space and one or more differently charged electrostatic attractor to draw the airborne antipathogenic agents through the meeting space;

x. ozone in the meeting space;

y. ozone in the meeting space and one or more positively charged electrostatic attractor to draw the ozone through the meeting space;

z. ionized air in the meeting space;

aa. one or more electrostatic attractor to draw ions and/or ionized airborne particles and/or pathogens and/or pathogens bound to ions through the meeting space;

bb. one or more electrostatic attractor to draw ions and/or ionized airborne particles and/or pathogens and/or pathogens bound to ions away from persons;

cc. devices that generate one or more airborne materials in the meeting space, the airborne materials for directly sterilizing the meeting space or acting as a carrier for another sterilant or anti-pathogen, or the airborne materials bind to pathogens inactivating them;

dd. one or more airborne materials in the meeting space, the airborne materials for directly sterilizing the meeting space or acting as a carrier for another sterilant or anti-pathogen, or the airborne materials bind to pathogens inactivating them, and means to move the airborne materials through the meeting space;

ee. IR from hot objects and/or fires and/or flames and/or IR sources and/or sunlight IR concentrated into the meeting space;

ff. airflows to remove pathogens from the meeting space;

gg. laminar airflows to remove pathogens from the meeting space;

hh. airflows and/or laminar airflows to move pathogens away from persons;

ii. airflows and/or laminar airflows that move pathogens in the meeting space away from persons;

jj. airflows and/or laminar airflows to move pathogens from persons into the meeting space;

kk. one or more electrostatic attractor which draws ions and/or ionized airborne particles and/or pathogens and/or pathogens bound to ions and/or ionized antipathogenic agents away from persons; and

ll. one or more electrostatic attractor which extracts and/or removes ions and/or ionized airborne particles and/or pathogens and/or pathogens bound to ions and/or ionized antipathogenic agents.

In some embodiments, the invention further comprises one or more adjacent person meeting spaces between adjacent persons that are not fully separated by at least one meeting space.

In some embodiments at least one partition between the adjacent persons is replaced by at least one adjacent person meeting space.

In some embodiments, the one or more adjacent person meeting spaces include the means to at least substantially reduce the number of pathogens in the adjacent person meeting space.

In some embodiments, the means to at least substantially reduce the number of pathogens further comprises means to substantially destroy and/or inactivate pathogens in the at least one adjacent person meeting space so that the pathogens cease to be infectious.

In some embodiments, where a space between adjacent persons using the meeting space exists, and means for destroying/inactivating/removing pathogens from the space between the adjacent persons not separated by the meeting space are provided, these means are the same as the means for destroying/inactivating/removing pathogens in the meeting space.

In some embodiments, where a space between adjacent persons using the meeting space exists, and means for destroying/inactivating/removing pathogens from the space between the adjacent persons not separated by the meeting space are provided, these means are different from the means for destroying/inactivating/removing pathogens in the meeting space and is not harmful to persons.

In some embodiments, the means to at least substantially reduce the number of pathogens from the at least one meeting space further comprises substantially removing the pathogens from the at least one adjacent person meeting space. In some embodiments the means is not harmful to the adjacent persons using the adjacent person meeting space and does not present a danger to them.

In some embodiments, the space between the adjacent persons serves as a meeting space.

In some embodiments, the means for destroying/inactivating or removing pathogens from the space between the adjacent persons and/or the means for destroying/inactivating or removing pathogens from the at least one meeting space is not harmful to persons.

In some embodiments, the means for destroying/inactivating or removing pathogens from the space between the adjacent persons and/or the means for destroying/inactivating or removing pathogens from the at least one meeting space is not harmful to persons.

Some embodiments of the invention comprise only one or more of the spaces between the adjacent persons, which serves as a meeting space; the space between the adjacent persons replaces the at least one central meeting space.

In some embodiments, the means for destroying/inactivating or removing pathogens is not harmful to persons and is one or more of the following:
1. A convective up-flow of air to remove pathogens;
2. A convective up-flow of air to remove pathogens and an exhaust;
3. Hot air of sufficient temperature to destroy or inactivate airborne pathogens without burning persons;
4. hot surfaces that generate infrared radiation for sterilization;
5. Safe ultraviolet light UV-C;
6. Sunlight concentrated in the meeting space and/or the space between the adjacent persons;
7. UV and/or IR radiation from sunlight;
8. Concentrated UV and/or IR from sunlight;
9. One or more electrostatic attractors to draw pathogens through the meeting space and/or the space between the adjacent persons;
10. one or more electrostatic attractors to draw pathogens away from persons;
11. one or more electrostatic attractors to attract pathogens where the attractor is heated to destroy pathogens on contact;
12. airborne ions that are not harmful to persons that bind to pathogens;
13. airborne negative ions that are not harmful to persons that bind to pathogens;
14. airborne ions and one or more differently charged electrostatic attractor to draw the airborne ions away from persons;
15. airborne ions and one or more differently charged electrostatic attractor to draw the airborne ions through the meeting space/and or the space between the adjacent persons;
16. ionized airborne antipathogenic agents that are not harmful to persons;
17. ionized airborne antipathogenic agents and one or more differently charged electrostatic attractor to draw the airborne ionized antipathogenic agents through the meeting space and/or the space between the adjacent persons;
18. ionized airborne antipathogenic agents and one or more differently charged electrostatic attractor to draw the airborne ionized antipathogenic agents away from persons;
19. ozone and one or more positively charged electrostatic attractor to draw the ozone through the meeting space and/or the space between the adjacent persons;
20. ozone and one or more positively charged electrostatic attractor to draw the ozone away from persons;
21. One or more electrostatic attractor to draw ions and/or ionized airborne particles and/or pathogens and/or pathogens bound to ions through the meeting space;
22. One or more electrostatic attractor to draw ions and/or ionized airborne particles and/or pathogens and/or pathogens bound to ions away from persons;
23. One or more electrostatic attractor to remove ions and/or ionized airborne particles and/or pathogens and/or pathogens bound to ions;
24. One or more airborne materials the airborne materials for directly sterilizing the meeting space and/or through the space between adjacent persons and/or acting as a carrier for another sterilant or anti-pathogen, or the airborne materials bind to pathogens inactivating them, where the airborne materials are not harmful to persons;
25. One or more airborne materials in the meeting space and/or the space between the adjacent persons, the airborne materials for directly sterilizing the space or acting as a carrier for another sterilant or anti-pathogen, or the airborne materials bind to pathogens inactivating them, and means to move the airborne materials through the meeting space and/or through the space between adjacent persons;
26. One or more airborne materials, the airborne materials for directly sterilizing the meeting space and/or the space between the adjacent persons or acting as a carrier for another sterilant or anti-pathogen, or the airborne materials bind to pathogens inactivating them, and means to move the airborne materials away from persons;
27. IR from hot objects and/or fires and/or flames and/or IR sources and/or sunlight IR;
28. airflows to remove pathogens;
29. laminar airflows to remove pathogens;
30. airflows and/or laminar airflows to move pathogens away from persons;
31. airflows and/or laminar airflows that move pathogens in the meeting space and/or in the space between the adjacent persons away from persons;
32. airflows and/or laminar airflows to move pathogens from persons into the meeting space and/or the space between the adjacent persons;
33. One or more electrostatic attractor which draws ions and/or ionized airborne particles and/or pathogens and/or pathogens bound to ions and/or ionized antipathogenic agents away from persons; and
34. One or more electrostatic attractor which extracts and/or removes ions and/or ionized airborne particles and/or pathogens and/or pathogens bound to ions and/or ionized antipathogenic agents.

In some embodiments, the means that is not harmful to persons is heated air of sufficient temperature to destroy pathogens and/or to extract pathogens by creating a convective updraft, without being of sufficient temperature to harm persons.

In some embodiments, persons are inside the one or more meeting space, wherein the means for substantial pathogen removal and/or destruction and/or inactivation are not harmful to those persons.

In some solar powered embodiments of the invention, the means is ultraviolet and/or infrared radiation, wherein the ultraviolet and/or infrared radiation is extracted from solar radiation and is used to substantially destroy and/or inactivate pathogens.

In some solar powered embodiments of the invention, the means comprises infrared radiation, the infrared radiation extracted from solar radiation and is used to destroy pathogens in the meeting space and/or to heat a heat source which heats air to substantially destroy pathogens and/or to create a convective airflow to remove pathogens from the one or more meeting space.

Some embodiments of the invention, utilize solar energy and further comprise one or more of the following:
1. Arrays of lenses and/or mirrors to focus sunlight into the meeting space and/or the space between adjacent participants;
2. Tracking means enabling the apparatus to track the position of the sun;
3. One or more target which becomes hot when it absorbs sunlight and/or IR from sunlight;
4. Lenses and/or mirrors to focus sunlight onto the absorbent target;
5. A target which absorbs sunlight and/or components of sunlight and becomes hot and creates a convective updraft in the one or meeting space and/or the space between adjacent persons;
6. A target which absorbs sunlight and/or components of sunlight and when it becomes hot and emits IR in the one or more meeting space and/or the space between adjacent persons;
7. The use of greenhouse materials including glass to increase the IR component of sunlight;
8. Means to store captured solar heat energy and to release it when sunlight is not available;
9. The use of solar heat energy and/or stored/released solar energy to create a convective updraft in the meeting space and/or the space between adjacent persons;
10. The use of solar heat energy and/or stored solar energy to vaporize an antipathogenic agent;
11. Reflectors enabling sunlight and/or concentrated sunlight in the meeting space and/or the space between the adjacent persons to be reflected back and/or concentrated into that space to increase its intensity;
12. The use of solar power to charge an electrostatic attractor; and
13. The use of solar power to ionize particles.

Some embodiments of the invention utilize solar energy and further comprise means to separate sunlight into UV, IR and Visible light to provide separate and complimentary means to substantially and/or completely destroy and/or inactivate and/or to remove pathogens.

Some embodiments of the invention use one or more of the following to separate sunlight into UV and/or IR and/or visible light:
a) materials that differentially reflect/transmit UV and/or IR and/or visible light;
b) materials that differentially refract UV and/or IR and/or visible light;
c) lenses that bring UV and/or IR and/or visible light to different focal points;
d) compound lenses made of different materials that bring UV and/or IR and/or visible light to different focal points;
e) Fresnel lenses that bring UV and/or IR and/or visible light to different focal points; and
f) compound Fresnel lenses made of different materials that bring UV and/or IR and/or visible light to different focal points.

Some embodiments of the invention that use solar energy, further provide means to generate and/or store electricity and/or static electricity which may be used for one or more of the following;

a) to charge electrostatic attractors;

b) to ionize particles;

c) to ionize air;

d) to generate ozone;

e) to ionize antipathogenic agents;

f) to power heating devices described herein;

g) to ionize and/or vaporize particles; and

h) to ionize and/or vaporize anti-pathogenic agents.

Some embodiments of the invention utilizing solar energy further provide means to generate and/or store electricity and/or static electricity by one or more of the following methods:
a) production of electricity and/or static electricity by the photoelectric effect;
b) the use of photovoltaic cells and arrays; and
c) known means to convert low voltage DC current from solar cells to high voltage electricity and static electricity.

In some embodiments of the invention, comprising means to extract UV from sunlight, the UV is used for one or more of the following:
a) to generate static energy by the photoelectric effect, which may be used to ionise an agent and/or to charge electrostatic attractor;
b) To provide a UV sterilisation beam;
c) to generate ozone as an antipathogenic agent;
d) UV extracted from sunlight in the meeting space and/or the space between the adjacent persons is reflected back and/or concentrated into that space to increase the intensity of UV in that space;
e) To create a pleasing visual effect to serve as a focus for conversation and/or discussion; and
f) To display subjects for discussion in the at least one meeting space.

In some embodiments of the invention, comprising means to extract IR from sunlight, the IR is used for one or more of the following:
a) to heat a heat absorbent target to produce a thermal up-flow;
b) to heat a heat absorbent target containing an anti-pathogenic agent that may be vaporized;
c) to heat a heat absorbent target containing an anti-pathogenic agent that may be vaporized and ionized; and
d) IR extracted from sunlight in the one or more meeting space and/or the space between the adjacent persons is reflected back and/or concentrated into that space to increase the intensity of IR in that space.

Some embodiments of the invention that utilise fires and/or flames and/or heat sources further provide IR reflectors to concentrate IR into the one or more meeting space and/or the space between adjacent persons.

Some embodiments of the invention that utilise fires and/or flames and/or heat sources and/or heated air further provide an exhaust to substantially and/or completely remove pathogens from the one or more meeting space and/or the space between adjacent persons.

In some embodiments of the invention, the means is at least one electrostatic attractor and/or at least one kind of airborne ion; so as to provide one or more type of airborne ion that binds to pathogens and/or one or more differently charged electrostatic attractor which extracts pathogens from the meeting space and/or draws pathogens away from persons, and/or the electrostatic attractor draws ions through the meeting space and/or moves them away from persons, and/or the electrostatic attractor extracts pathogens bound to ions from the meeting space and/or moves pathogens bound to ions away from persons.

Some embodiments of the invention further comprise the use of sound reflectors to enhance communication between the persons.

In some embodiments of the invention, sound reflectors are used to direct sound from persons into the meeting space and/or to direct sound leaving the meeting space towards persons.

In some embodiments of the invention the sound reflectors are concave and/or parabolic and/or person's heads are at the focal point of the sound reflectors.

Some embodiments of the invention use sound reflectors to increase the social distance between persons and therefore do not need a meeting space with sterilization/extraction means.

Some embodiments of the invention further comprise means to provide persons with fresh air and/or to move the fresh air through the at least one meeting space.

In some embodiments of the invention, the means to substantially and/or completely destroy and/or inactivate and/or remove pathogens is used to create a pleasing visual effect which serves as a focus for conversation and/or discussion.

In some embodiments of the invention, the means to substantially and/or completely destroy and/or inactivate and/or remove pathogens is used to display a subject for discussion in the meeting space.

In some embodiments of the invention, an electrostatic field is used to substantially destroy/inactivate and/or remove pathogens and also to create a pleasing visual image in a chamber containing fluorescent gas which serves as a focus for conversation, and a scanning laser and/or a projector is used to project a subject for discussion into the chamber.

In some embodiments of the invention, a fire and/or a flame is used to substantially destroy/inactivate and/or remove pathogens and a scanning laser and/or a projector is used to project a subject for discussion into the fire and/or flame.

In some embodiments of the invention, UV is used to substantially destroy/inactivate and/or remove pathogens and also to create a pleasing visual image which serves as a focus for discussion and/or conversation.

In some embodiments of the invention, UV is used to substantially destroy/inactivate and/or remove pathogens and also to cause a subject for discussion to be displayed in visible light.

In some embodiments of the invention sunlight is used to substantially destroy/inactivate and/or remove pathogens and also to display a subject for discussion.

In some embodiments of the invention, airborne antipathogenic agents and/or ions are used to substantially destroy/inactivate and/or remove pathogens and also to create a pleasing visual image which serves as a focus for discussion and/or conversation.

In some embodiments of the invention, the means to substantially destroy/inactivate and/or remove pathogens are concentrated around the perimeter of the meeting space and/or the outer section of the meeting space closest to persons using the meeting space.

The invention includes the method of using the apparatus of the invention to safely accommodate one or more of meetings, face-to-face conversation, discussion, networking events, business meetings, focus groups, conferences, conventions, discussion forums, discussion groups and educational activities, social events involving conversation or discussion between persons.

The invention includes the commercial use of the apparatus of the invention by businesses and/or organizations to safely accommodate one or more of meetings, face-to-face conversation, discussion, networking events, business meetings, focus groups, conferences, conventions, discussion forums, discussion groups and educational activities, social events involving conversation or discussion between persons.

The invention includes the method of using the one or more meeting space of the invention for one or more of the following:
1. Advertising or promotional or sales events and/or advertising or promotional or sales events that involve face-to-face conversation
2. Meetings for the provision of business services or customer services;
3. Events for the provision of financial services;
4. Focus groups, and optionally creating focus groups for market research or for product development or for political organizations;
5. Political events;
6. Cultural events;
7. Conventions;
8. book fairs;
9. trade shows;
10. community events;
11. civic events;
12. personal development events;
13. professional development events;
14. improving communication within companies or organizations;
15. improving communication between companies or between organizations;
16. group therapy or other therapeutic use based on face-to-face conversation;
17. medical or therapeutic use by providing social interaction;
18. medical or therapeutic use by providing social connection;
19. medical or therapeutic use by connecting patient groups with related medical conditions;
20. medical or therapeutic use by sharing experiences of medical conditions,
21. educational use to teach or learn communication skills or networking skills or social skills;
22. educational use that involves teaching or learning by face-to-face discussion
23. recruitment events;
24. matching job applicants to employers;
25. networking between job applicants and employers;
26. mediation events, mediation between groups or individuals;
27. conflict resolution events;
28. dating events,
29. networking events arising from conferences or conventions; and networking events arising from conferences or conventions involving the creation of breakout groups,
30. business networking,
31. social networking,
32. professional career networking,
33. expert networking,
34. expert networking in areas including medicine and related professions, science, biotechnology, pharmaceuticals, technology, engineering, computing, electronics, digital technology, and
35. a musical performance.

In some embodiments the invention is a system providing one or more safe meeting spaces for two or more persons, wherein

a) the system is configured so that persons are outside the one or more meeting spaces and persons can communicate through the one or more meeting spaces;

b) the system is configured so that pathways for the possible spread of pathogens between persons pass through the one or more meeting spaces;

c) means are provided to make the one or more meeting spaces sterile or to substantially reduce the number of pathogens in the one or more meeting spaces;

d) the system is configured so that sound may pass through the one or more meeting spaces thereby enabling persons to hear each other; and

e) the system is configured so that at least two or more persons may see each other through the one or more meeting spaces.

In some embodiments the invention is a system providing a safe central meeting space for two or more persons wherein:

a) the system is configured to separate and space apart the persons to establish the central meeting space there between, so that persons are outside the central meeting space and persons can communicate through the central meeting space;

b) the system is configured so that pathways for the possible spread of pathogens between persons using the central meeting space, pass through the central meeting space by providing partitions positioned between adjacent persons using the central meeting space;

c) the partitions between the adjacent persons using the central meeting space are positioned outside of the central meeting space so that pathogens cannot pass directly between the adjacent persons, and so that pathways for the possible spread of pathogens between the adjacent persons pass through the central meeting space;

d) means are provided to make the central meeting spaces sterile or to substantially reduce the number of pathogens in the central meeting space;

e) the system is configured so that sound may pass through the central meeting space thereby enabling persons to hear each other; and

f) the system is configured so that at least two or more persons may see each other through the central meeting space.

In some embodiments the invention is a system providing one or more safe meeting space for two or more persons wherein:

a) the system is configured to separate and space apart the persons to establish a central meeting space there between, so that persons are outside the central meeting space and persons can communicate through the central meeting space;

b) the system is configured to provide further meeting spaces between adjacent persons using the central meeting space, so that the adjacent persons are outside the adjacent person meeting space and adjacent persons can communicate through the adjacent person meeting space;

c) the system is configured so that pathways for the possible spread of pathogens between persons pass through the central meeting space and/or through one or more of the adjacent person meeting spaces;

d) means are provided to make the central meeting space sterile or to substantially reduce the number of pathogens in the central meeting space;

e) means are provided to make the one or more adjacent person meeting spaces sterile or to substantially reduce the number of pathogens in the one or more adjacent person meeting spaces;

f) the system is configured so that sound may pass through the central meeting space and through the one or more adjacent person meeting spaces thereby enabling persons to hear each other; and

g) the system is configured so that at least two or more persons may see each other through the central meeting space and/or through the one or more adjacent person meeting spaces.

In some embodiments the invention provides one or more central meeting spaces.

In some embodiments of the invention all pathways for the possible spread of pathogens between persons pass through the one or more meeting spaces.

In some embodiments of the invention substantially all pathways for the possible spread of pathogens between persons pass through the one or more meeting spaces.

In some embodiments of the invention the shortest pathways for the possible spread of pathogens between persons pass through the one or more meeting spaces.

In some embodiments of the invention all pathways for the possible spread of pathogens between persons within the perimeter of the invention pass through the one or more meeting spaces.

In some embodiments of the invention substantially all pathways for the possible spread of pathogens between persons within the perimeter of the invention pass through the one or more meeting spaces.

In some embodiments of the invention the shortest pathways for the possible spread of pathogens between persons within the perimeter of the invention pass through the one or more meeting spaces.

In some embodiments of the invention substantially all pathways for the possible spread of pathogens between persons using the same meeting space pass through the same meeting space.

In some embodiments of the invention substantially all pathways for the possible spread of pathogens between persons using the same one or more meeting spaces pass through the same one or more meeting spaces.

In some embodiments the invention is a communication system.

In some embodiments the invention is a communication system providing one or more safe meeting spaces for two or more persons, wherein

a) the communication system is configured so that persons are outside the one or more meeting spaces and persons can communicate through the one or more meeting spaces;

b) the communication system is configured so that pathways for the possible spread of pathogens between persons pass through the one or more meeting spaces;

c) means are provided to make the one or more meeting spaces sterile or to substantially reduce the number of pathogens in the one or more meeting spaces;

d) the communication system is configured so that sound may pass through the one or more meeting spaces thereby enabling persons to hear each other; and

e) the communication system is configured so that at least two or more persons may see each other through the one or more meeting spaces.

means are provided to make the central meeting space sterile or to substantially reduce the number of pathogens in the central meeting space.

In some embodiments of the invention the means to reduce the number of pathogens in the one or more meeting space reduces the number of pathogens by one or more of the following:
a) the means reduces the number of pathogens by at least 10% per minute
b) the means reduces the number of pathogens by at least 20% per minute
c) the means reduces the number of pathogens by at least 30% per minute
d) the means reduces the number of pathogens by at least 40% per minute
e) the means reduces the number of pathogens by at least 45% per minute
f) the means reduces the number of pathogens by at least 50% per minute
g) the means reduces the number of pathogens by at least 60% per minute
h) the means reduces the number of pathogens by at least 70% per minute
i) the means reduces the number of pathogens by at least 80% per minute
j) the means reduces the number of pathogens by at least 90% per minute
k) the means reduces the number of pathogens by at least 95% per minute
l) the means reduces the number of pathogens by at least 99% per minute
m) the means reduces the number of pathogens by at least 99.5% per minute
n) the means reduces the number of pathogens by at least 99.9% per minute
o) the means reduces the number of pathogens by at least 99.99% per minute
p) the means reduces the number of pathogens by at least 99.999% per minute
q) the means reduces the number of pathogens by at least 99.9999% per minute

In some embodiments of the invention the means to reduce the number of pathogens in the one or more meeting space reduces the number of pathogens by one or more of the following:
a) the means reduces the number of pathogens by at least 50% per hour
b) the means reduces the number of pathogens by at least 60% per hour
c) the means reduces the number of pathogens by at least 70% per hour
d) the means reduces the number of pathogens by at least 80% per hour
e) the means reduces the number of pathogens by at least 90% per hour
f) the means reduces the number of pathogens by at least 95% per hour
g) the means reduces the number of pathogens by at least 99% per hour
h) the means reduces the number of pathogens by at least 99.5% per hour
i) the means reduces the number of pathogens by at least 99.9% per hour
j) the means reduces the number of pathogens by at least 99.99% per hour
k) the means reduces the number of pathogens by at least 99.999% per hour

In some embodiments of the invention the means destroys and/or inactivates substantially all pathogens in the one or more meeting spaces.

In some embodiments of the invention the means removes substantially all pathogens from the one or more meeting spaces.

In some embodiments, the invention is a system providing one or more safe meeting spaces for two or more persons, wherein

a) the system is configured so that persons are outside the one or more meeting spaces and persons can communicate through the one or more meeting spaces;

b) the system is configured so that substantially all pathways for the possible spread of pathogens between persons within the perimeter of the invention pass through the one or more meeting spaces.

c) means are provided to destroy and/or to inactivate and/or to remove substantially all pathogens in the one or more meeting spaces;

d) the system is configured so that sound may pass through the one or more meeting spaces thereby enabling persons to hear each other; and

e) the system is configured so that at least two or more persons may see each other through the one or more meeting spaces.

In some embodiments the invention is a system providing one or more safe meeting spaces for two or more persons, wherein

a) the system is configured so that persons are outside the one or more meeting spaces and persons can communicate through the one or more meeting spaces;

b) the system is configured so that substantially all pathways for the possible spread of pathogens between persons using the same one or more meeting spaces pass through the same one or more meeting spaces.

c) means are provided to destroy and/or inactivate and/or to remove substantially all pathogens in the one or more meeting spaces;

d) the system is configured so that sound may pass through the one or more meeting spaces thereby enabling persons to hear each other; and

e) the system is configured so that at least two or more persons may see each other through the one or more meeting spaces.

In some embodiments the invention is a system providing one or more safe meeting spaces for two or more persons, wherein

a) the system is configured so that persons are outside the one or more meeting spaces and persons can communicate through the one or more meeting spaces;

b) the system is configured so that substantially all pathways for the possible spread of pathogens between persons using the same one or more meeting spaces pass through the same one or more meeting spaces.

c) means are provided to destroy and/or to inactivate and/or to remove substantially all pathogens in the one or more meeting spaces;

d) the system is configured so that sound may pass through the one or more meeting spaces thereby enabling persons using the same one or more meeting spaces to hear each other;

e) the system is configured so that at least two or more persons using the same one or more meeting spaces may see each other through the one or more meeting spaces; and

f) the system is configured to prevent substantially all pathogens passing between persons using different meeting spaces and/or means are provided to prevent substantially all pathogens passing between persons using different meeting spaces.

In some embodiments means are provided to prevent pathogens passing between persons using different meeting spaces.

In some embodiments barriers are provided to prevent pathogens passing between persons using different meeting spaces.

In some embodiments heating systems are utilized to reduce the possible spread of pathogens passing between persons using different meeting spaces.

In some embodiments cooling systems are utilized to reduce the possible spread of pathogens passing between persons using different meeting spaces.

In some embodiments ventilation systems are utilized to reduce the possible spread of pathogens passing between persons using different meeting spaces.

In some embodiments some or all pathogens from persons using a meeting space are drawn into that meeting space to prevent them from entering the space where the invention is located.

In some embodiments most pathogens from persons using a meeting space are drawn into that meeting space to prevent them from entering the space where the invention is located.

In some embodiments substantially all pathogens from persons using a meeting space are drawn into that meeting space to prevent them from entering the space where the invention is located.

In some embodiments pathogens from persons using the one or more meeting spaces are drawn into those meeting spaces by one or more of the following means:
a) By extracting air from the meeting space(s)
b) By venting air from the meeting space(s)
c) By using an extractor fan to extract air from the meeting space(s)
d) By creating negative air pressure in the meeting space(s) in relation to the external space in which persons using that meeting space are located
e) By creating positive air pressure in the space in which persons are located in relation to the one or more meeting spaces
f) By providing clean air flows to persons using the meeting space(s) and creating positive air pressure in the space in which persons are located in relation to the one or more meeting spaces
g) By creating a convective updraft in the one or more meeting spaces
h) By providing an electrostatic attractor in the one or more meeting spaces to attract pathogens into the one or more meeting spaces
i) By negatively ionising the space in which persons are located and providing a positively charged electrostatic attractor in the one or more meeting spaces to draw ions
j) Various other means described herein to facilitate the flow of pathogens from persons into the one or more meeting spaces.

In some embodiments the system is configured to prevent substantially all pathogens passing between persons using different meeting spaces and/or means are provided to prevent substantially all pathogens passing between persons using different meeting spaces.

In some embodiments the system is configured to substantially reduce the number of pathogens passing between persons using different meeting spaces and/or means are provided to substantially reduce the number of pathogens passing between persons using different meeting spaces.

In some embodiments the system is configured to reduce the number of pathogens passing between persons using different meeting spaces and/or means are provided to reduce the number of pathogens passing between persons using different meeting spaces.

In some embodiments the invention is a system providing one or more safe meeting spaces for two or more persons, wherein

a) the system is configured so that persons are outside the one or more meeting spaces and persons can communicate through the one or more meeting spaces;

b) the system is configured so that substantially all pathways for the possible spread of pathogens between persons using the same one or more meeting spaces pass through the same one or more meeting spaces.

c) means are provided to destroy and/or inactivate and/or to remove substantially all pathogens in the one or more meeting spaces;

d) the system is configured so that sound may pass through the one or more meeting spaces thereby enabling persons to hear each other; and

e) the system is configured so that at least two or more persons may see each other through the one or more meeting spaces.

In some embodiments the invention is a system providing one or more safe central meeting spaces for two or more persons, wherein

a) the system is configured so that persons are outside the one or more central meeting spaces and persons can communicate through the one or more central meeting spaces;

b) the system is configured so that substantially all pathways for the possible spread of pathogens between persons using the same one or more central meeting spaces pass through the same one or more meeting spaces.

c) means are provided to destroy and/or inactivate and/or to remove substantially all pathogens in the one or more central meeting spaces;

d) the system is configured so that sound may pass through the one or more meeting spaces thereby enabling persons using the same central meeting space to hear each other; and

e) the system is configured so that at least two or more persons using the same central meeting space may see each other through the same central meeting spaces.

In some embodiments the invention is a system providing one or more safe meeting space for two or more persons wherein:

a) the system is configured to separate and space apart the persons to establish one or more central meeting spaces there between, so that persons are outside the one or more central meeting spaces and persons can communicate through the one or more central meeting spaces;

b) the system is configured to provide further meeting spaces between adjacent persons using the same central meeting space, so that the adjacent persons are outside the adjacent person meeting space and adjacent persons can communicate through the adjacent person meeting spaces;

c) the system is configured so that substantially all pathways for the possible spread of pathogens between persons using the same central meeting space pass through the same central meeting space and/or through one or more of the adjacent person meeting spaces;

d) means are provided to make the one or more central meeting spaces sterile or to substantially reduce the number of pathogens in the one or more central meeting spaces;

e) means are provided to make the one or more adjacent person meeting spaces sterile or to substantially reduce the number of pathogens in the one or more adjacent person meeting spaces;

f) the system is configured so that sound may pass through the one or more central meeting space and through the one or more adjacent person meeting spaces thereby enabling persons using the same meeting space to hear each other; and

g) the system is configured so that at least two or more persons using the same meeting space may see each other through the central meeting space and/or through the one or more adjacent person meeting spaces.

In some embodiments the invention provides one or more central meeting spaces.

In some embodiments the means to destroy and/or to inactivate and/or to remove pathogens is also used to heat buildings and/or to heat outdoor spaces. The provision of one or more safe meeting spaces together with means to heat buildings and/or outdoor spaces is a further advantage of the invention.

A further feature of some embodiments is the use of heat energy and/or heated air from the meeting space(s) to heat buildings or outdoor spaces. The dual efficiency of using the same heat energy to create the means and also to heat buildings, is an important advantage of some embodiments the invention.

In some embodiments, the means to heat buildings and/or to heat outdoor spaces reduces the possible spread of pathogens between persons using different meeting spaces. In some embodiments, the means to heat buildings and/or to heat outdoor spaces substantially reduces the possible spread of pathogens between persons using different meeting spaces. In some embodiments the means to heat buildings and/or to heat outdoor spaces prevents the possible spread of substantially all pathogens between persons using different meeting spaces.

In some embodiments, heat and/or heated air from the one or more meeting spaces is used to heat buildings and/or to heat outdoor spaces where the invention is situated. Provided the air is of sufficiently high temperature to destroy pathogens therein, it can be extracted/collected and used to heat the buildings and/or outdoor spaces. See FIG. 11.

In some embodiments, air extracted from the one or more meeting spaces is at typical room temperature but its expulsion from the building would cause significant heat loss to the building during cold weather. In some of these embodiments, the room temperature air extracted from the one or more meeting spaces may be further heated to destroy pathogens or to destroy substantially all pathogens, before being used to heat the building. In some of these embodiments, the room temperature air extracted from the one or more meeting spaces may be filtered using known means to remove pathogens or to remove substantially all pathogens, before being returned to the building.

In some embodiments, air in the one or more meeting spaces is heated to sufficient temperature to create a convective up-flow to remove pathogens from the one or more meeting spaces but not to a sufficiently high temperature to destroy all pathogens or to destroy substantially all pathogens. In some embodiments, air in the one or more meeting spaces is heated to sufficient temperature to destroy some or most pathogens but is not hot enough to harm persons using the one or more heating space. In some embodiments, the insufficiently heated air from the one or more meeting spaces is further heated to a sufficiently high temperature to destroy all pathogens therein or to destroy substantially all pathogens therein, before being used to heat the building. In some embodiments, the insufficiently heated air from the one or more meeting spaces may be filtered using known means to remove pathogens, before being returned to the building.

In some embodiments, air from the one or more meeting spaces and/or heated air from the one or more meeting spaces is further heated to a sufficiently high temperature to ensure that pathogens are destroyed or inactivated before it is used to heat buildings or to heat outdoor spaces. In some embodiments, air from the one or more meeting spaces and/or heated air from the one or more meeting spaces is filtered using known means to remove pathogens and/or it is further heated to sufficient temperature to destroy and/or to remove all or substantially all pathogens, before being used to heat buildings and/or outdoor spaces. See FIG. 12.

In some embodiments, heated air from the one or more meeting spaces which may contain pathogens is used to heat clean air or to heat water via one or more heat exchanger or heat pump. The heated clean air or hot water leaving the one or more heat exchanger or heat pump may then be used to heat buildings or outdoor spaces. In some embodiments, air from the one or more meeting spaces leaving the heat exchanger or heat pump is vented away (see FIG. 13). In some embodiments air from the space in which the invention is located is passed through one or more heat exchangers or heat pumps to be heated and it is then used to heat buildings or to heat outdoor spaces. In some embodiments air from the space in which the invention is located is filtered to remove pathogens before being passed through the one or more heat exchangers/heat pumps and is then used to heat buildings or outdoor spaces. In some embodiments the air from the space in which the invention is located is further heated after passing through the one or more heat exchangers/heat pumps to destroy pathogens before being used to heat buildings and/or outdoor spaces. See FIG. 14. This sort of embodiment provides means to remove pathogens from the space in which it is located, while also heating that space. This sort of embodiment provides means to reduce the spread of pathogens between persons using different meeting spaces.

In some embodiments, the means to destroy and/or to inactivate and/or to remove pathogens is also used to cool buildings and/or to cool outdoor spaces. In some embodiments the means to destroy and/or to inactivate and/or to remove pathogens is also used to increase the efficiency of the refrigeration cycle of air conditioning systems.

In some embodiments, an extractor fan is placed above the one or more meeting spaces so that pathogens are drawn up into the one or more meeting space and away from persons.

In some embodiments, air extracted from the one or more meeting spaces is used to cool the condenser coil of an air-conditioning unit/refrigeration unit, before being vented away. In some embodiments, air from the place in which the invention is located is drawn into the air conditioning/refrigeration unit and is cooled by the evaporator coil. The cooled air may also be filtered to remove pathogens before being returned to the place in which the invention is located. This sort of embodiment provides means to remove pathogens from the space in which it is located, while also cooling that space. This sort of embodiment provides means to reduce the spread of pathogens between persons using different meeting spaces. The use of relatively cool air extracted from the one or more meeting spaces to cool the condenser coil increases the efficiency of the refrigeration cycle, as compared to the use of warmer air from outside the building where the invention is located. This mitigates the loss of unclean cool air vented away from the place in which the invention is located (see FIG. 15). In some embodiments, in airconditioned spaces, air extracted from the one or more meeting spaces is filtered to remove pathogens before being returned to the space in which the invention is located.

Buildings need to be heated so it is highly efficient to use the same energy to create safe meeting spaces. A further feature of some embodiments is the use of heat energy and/or heated air from the meeting space(s) to heat buildings or outdoor spaces. The dual efficiency of using the same heat energy to create safe meeting spaces and also to provide heating, is an important advantage of some embodiments the invention. Further additional features increase energy efficiency, such as recycling heat energy from the meeting space back into the meeting space.

In some embodiments, the heat energy that creates the safe meeting space(s) is also used to heat buildings or outdoor spaces.

In some embodiments, the heat energy that creates the safe meeting space(s) is recycled back into the meeting space(s).

In some embodiments, the heat energy that creates the safe meeting space(s) is used to heat clean (pathogen-free) air which is recycled back into the meeting space(s).

In some embodiments, the heat energy that creates the safe meeting space(s) is also used to heat clean (pathogen-free) air, which is supplied to persons in person locations, increasing the positive pressure of the person locations in relation to the meeting space(s) thereby increasing the flow of pathogens from the person locations into the meeting space(s).

In some embodiments, the heat energy that creates the safe meeting space(s) is also used to provide warm clean (pathogen-free) air, which is supplied to persons in person locations, increasing the positive pressure of the person locations in relation to the meeting space(s) thereby increasing the flow of pathogens from the person locations into the meeting space(s).

In some embodiments, the heat energy that creates the safe meeting space(s) is also used to provide warm clean (pathogen-free) air, which is supplied to persons in person locations, increasing the positive pressure of the person locations in relation to the meeting space(s) thereby increasing the flow of pathogens from the person locations into the meeting space(s).

In some embodiments, the heat energy that creates the safe meeting space(s) is also used to provide warm clean (pathogen-free) air, which is supplied to the zone surrounding person locations, increasing the positive pressure around person locations and increasing the flow of air into person locations and also increasing the flow of pathogens from the person locations into the meeting space(s). This sort of embodiment also reduces the flow of pathogens from person locations into the space where the meeting space(s) are located.

In some embodiments, the heat energy that creates the safe meeting space(s) is also used to heat buildings or outdoor spaces.

In some embodiments, the heat energy that provides the means is recycled back into the meeting space(s).

In some embodiments, the heat energy that provides the means is also used to heat clean (pathogen-free) air which is recycled back into the meeting space(s).

In some embodiments, the heat energy that provides the means is also used to heat clean (pathogen-free) air, which is supplied to persons in person locations, increasing the positive pressure of the person locations in relation to the meeting space(s) thereby increasing the flow of pathogens from the person locations into the meeting space(s).

In some embodiments, the heat energy that provides the means is also used to provide warm clean (pathogen-free) air, which is supplied to persons in person locations, increasing the positive pressure of the person locations in relation to the meeting space(s) thereby increasing the flow of pathogens from the person locations into the meeting space(s).

In some embodiments, the heat energy that provides the means is also used to provide warm clean (pathogen-free) air, which is supplied to persons in person locations, increasing the positive pressure of the person locations in relation to the meeting space(s) thereby increasing the flow of pathogens from the person locations into the meeting space(s).

In some embodiments, the heat energy that provides the means is also used to provide warm clean (pathogen-free) air, which is supplied to the zone surrounding person locations, increasing the positive pressure around person locations and increasing the flow of air into person locations and thereby increasing the flow of pathogens from the person locations into the meeting space(s). This sort of embodiment also reduces the flow of pathogens from person locations into the space where the meeting space(s) are located. This sort of embodiment reduces the spread of pathogens between persons using different meeting spaces.

In some embodiments, cool clean (pathogen-free) air is supplied to the zone surrounding person locations, increasing the positive pressure around person locations and increasing the flow of air into person locations and thereby increasing the flow of pathogens from the person locations into the meeting space(s). This sort of embodiment also reduces the flow of pathogens from person locations into the space where the meeting space(s) are located. This sort of embodiment reduces the spread of pathogens between persons using different meeting spaces.

In some embodiments, the meeting space(s) have extraction means and are at negative pressure in relation to the person locations. In these embodiments the meeting space(s) act as wells for the extraction of pathogens from the space in which they are located.

In some embodiments, the meeting space(s) have extraction means and are at negative pressure in relation to the space in which they are located. In these embodiments the meeting space(s) act as wells for the extraction of pathogens from the space in which they are located.

In some embodiments, plants are used synergistically with other sterilisation means/pathogen extraction means to further remove and/or absorb pathogens and/or to capture carbon and/or to provide fresh oxygen and/or for cooling.

In some embodiments, plants are used to remove airborne pathogens. In some embodiments plants are used to remove airborne pathogens and/or to capture carbon from CO₂ produced by persons. In some embodiments plants are used to remove airborne pathogens and/or to capture carbon from CO₂ produced by persons and/or to produce fresh oxygen. In some embodiments, plants are used to cool the place where the invention is located by transpiration/evaporation. In some embodiments plants are used to create an up-flow of air by transpiration/evaporation to remove pathogens.

In some embodiments, plants are used to remove airborne pathogens and/or to capture carbon from CO₂ produced by persons and/or to produce fresh oxygen and/or to cool the place where the invention is located by transpiration/evaporation and/or to create an up-flow of air by transpiration/evaporation to remove pathogens.

In some embodiments, plants are used to absorb pathogens/remove pathogens from the space between different meeting spaces to reduce the spread of pathogens between different meeting spaces. In some embodiments plants are used to absorb pathogens in the one or more meeting spaces and/or to remove pathogens from the one or more meeting spaces by creating an up-flow in the one or more meeting spaces through transpiration and evaporation.

In some embodiments, an electrostatic charge is applied to plants to further attract airborne pathogens and/or to increase the rate of plant growth and/or to increase the rate of photosynthesis for carbon capture and/or for fresh oxygen production.

Some solar embodiments of the invention separately extract IR, UV and visible light from sunlight. In some solar embodiments the IR and UV components are used to destroy and/or to inactivate and/or to remove pathogens, while visible light extracted from sunlight may be used to promote plant growth to further absorb/remove pathogens and/or to increase the rate of plant growth and/or to increase the rate of photosynthesis for carbon capture and/or for fresh oxygen production and/or for cooling.

In some embodiments, the invention is a system providing one or more safe meeting spaces for two or more persons wherein:

a) the system provides a central meeting space and person locations outside the central meeting space for persons using the central meeting space, with no barriers between the central meeting space and the person locations such that persons in the person locations can communicate with each other through the central meeting space,

b) outside the central meeting space and between adjacent person locations, are provided adjacent person meeting spaces or partitions,

c) the system is configured such that substantially all pathways within the system for the possible spread of pathogens between the person locations pass through at least one of the meeting spaces or are blocked by a partition,

d) means are provided specifically to make the meeting space(s) sterile or to substantially reduce the number of pathogens in the meeting spaces(s),

e) the system is configured to allow for persons in the person locations using the same meeting space(s) to hear each other through the meeting spaces(s) and for at least two persons using the same meeting space(s) to see each other through the meeting spaces(s).

In some embodiments, the invention is a system to provide one or more safe meeting spaces for two or more persons wherein:
a) the system provides a central meeting space and person locations outside the central meeting space for persons using the system, with no barriers between the central meeting space and the person locations such that persons in the person locations can communicate with each other through the central meeting space,
b) outside the central meeting space and between adjacent person locations, are provided adjacent person meeting spaces or partitions,
c) the system is configured such that all pathways for possible spread of pathogens between the person locations pass through at least one of the meeting spaces or are blocked by a partition,
d) means are provided to make only the meeting space(s) sterile or to substantially reduce the number of pathogens in the meeting spaces(s),
e) the system is configured to allow for persons in the person locations to see and hear each other through the meeting spaces(s).

In some embodiments, the invention is a system to provide one or more safe meeting spaces for two or more persons wherein:
a) the system provides a central meeting space and person locations outside the central meeting space for persons using the system, with no barriers between the central meeting space and the person locations such that persons in the person locations can communicate with each other through the central meeting space,
b) outside the central meeting space and between adjacent person locations, are provided adjacent person meeting spaces or partitions,
c) the system is configured such that all pathways for possible spread of pathogens between the person locations pass through at least one of the meeting spaces or are blocked by a partition,
d) means are provided specifically to make the meeting space(s) sterile or to substantially reduce the number of pathogens in the meeting spaces(s),
e) the system is configured to allow for persons in the person locations to see and hear each other through the meeting spaces(s).

In some embodiments, system to provide one or more safe meeting spaces for two or more persons wherein:
a) the system provides a central meeting space and person locations outside the central meeting space for persons using the system, with no barriers between the central meeting space and the person locations such that persons in the person locations can communicate with each other through the central meeting space,
b) outside the central meeting space and between adjacent person locations, are provided adjacent person meeting spaces or partitions,
c) the system is configured such that all pathways within the system for possible spread of pathogens between the person locations pass through at least one of the meeting spaces or are blocked by a partition,
d) means are provided to make the meeting space(s) sterile or to substantially reduce the number of pathogens in the meeting spaces(s), wherein the means are specifically required to make the meeting space(s) sterile or to substantially reduce the number of pathogens in the meeting spaces(s),
e) the system is configured to allow for persons in the person locations using the same meeting space(s) to hear each other through the meeting spaces(s) and for at least two persons using the same meeting space(s) to see each other through the meeting spaces(s).

In some embodiments, system to provide more than one safe meeting space for two or more persons

wherein:

a) the system provides more than one meeting space and person locations outside the meeting spaces for persons using the system, with no barriers between the meeting space(s) and the person locations such that persons in the person locations can communicate with each other through the meeting space(s),

b) the system is configured such that all pathways within the system for possible spread of pathogens between the person locations pass through at least one of the meeting spaces or are blocked by a barrier,

c) means are provided to make the meeting space(s) sterile or to substantially reduce the number of pathogens in the meeting spaces(s), wherein the means are specifically required to make the meeting space(s) sterile or to substantially reduce the number of pathogens in the meeting spaces(s),

d) the system is configured to allow for persons in the person locations using the same meeting space(s) to hear each other through the meeting spaces(s) and for at least two persons using the same meeting space(s) to see each other through the meeting space(s).

In some embodiments, the invention is a system providing more than one safe meeting space for two or more persons wherein:

a) the system provides more than one meeting space and person locations outside the meeting spaces for persons using the meeting spaces, with no barriers between the meeting space(s) and the person locations such that persons in the person locations using the same meeting space(s) can communicate with each other through the meeting space(s),

b) the system is configured such that substantially all pathways within the system for the possible spread of pathogens between the person locations pass through at least one of the meeting spaces or are blocked by a barrier,

c) means are provided specifically to make the meeting space(s) sterile or to substantially reduce the number of pathogens in the meeting spaces(s),

d) the system is configured to allow for persons in the person locations using the same meeting space(s) to hear each other through the meeting spaces(s) and for at least two persons using the same meeting space(s) to see each other through the meeting space(s).

In some embodiments, the invention is a system providing more than one safe meeting space for two or more persons wherein:

a) the system provides more than one meeting space and person locations outside the meeting spaces for persons using the meeting spaces, with no barriers between the meeting space(s) and the person locations such that persons in the person locations using the same meeting space(s) can communicate with each other through the meeting space(s),

b) the system is configured such that substantially all pathways within the system for the possible spread of pathogens between persons using the same meeting spaces pass through at least one of the meeting spaces or are blocked by a barrier,

c) means are provided to make the meeting space(s) sterile or to substantially reduce the number of pathogens in the meeting spaces(s),

d) the system is configured to allow for persons in the person locations using the same meeting space(s) to hear each other through the meeting spaces(s) and for at least two persons using the same meeting space(s) to see each other through the meeting space(s).

In some embodiments, the invention is a system comprising more than one system as described here.

In some embodiments, the invention is a system providing one or more safe meeting spaces for two or more persons wherein:
a) the system provides a central meeting space and person locations outside the central meeting space for persons using the central meeting space, with no barriers between the central meeting space and the person locations such that persons in the person locations can communicate with each other through the central meeting space,
b) outside the central meeting space and between adjacent person locations, are provided adjacent person meeting spaces or partitions,
c) the system is configured such that substantially all pathways within the system for the possible spread of pathogens between the person locations pass through at least one of the meeting spaces,
d) means are provided to make the meeting space(s) sterile or to substantially reduce the number of pathogens in the meeting spaces(s),
e) the system is configured to allow for persons in the person locations using the same meeting space(s) to hear each other through the meeting spaces(s) and for at least two persons using the same meeting space(s) to see each other through the meeting spaces(s).

In some embodiments, the invention is a system providing more than one safe meeting space for two or more persons wherein:
a) the system provides more than one meeting space and person locations outside the meeting spaces for persons using the meeting spaces, with no barriers between the meeting space(s) and the person locations such that persons in the person locations using the same meeting space(s) can communicate with each other through the meeting space(s),
b) the system is configured such that substantially all pathways within the system for the possible spread of pathogens between the person locations pass through at least one of the meeting spaces,
c) means are provided to make the meeting space(s) sterile or to substantially reduce the number of pathogens in the meeting spaces(s),
d) the system is configured to allow for persons in the person locations using the same meeting space(s) to hear each other through the meeting spaces(s) and for at least two persons using the same meeting space(s) to see each other through the meeting space(s).

In some embodiments, the invention is a system providing more than one safe meeting space for two or more persons wherein:
a) the system provides more than one meeting space and person locations outside the meeting spaces for persons using the meeting spaces, with no barriers between the meeting space(s) and the person locations such that persons in the person locations using the same meeting space(s) can communicate with each other through the meeting space(s),
b) the system is configured such that substantially all pathways within the system for the possible spread of pathogens between persons using the same meeting spaces pass through at least one of the meeting spaces,
c) means are provided to make the meeting space(s) sterile or to substantially reduce the number of pathogens in the meeting spaces(s),
d) the system is configured to allow for persons in the person locations using the same meeting space(s) to hear each other through the meeting spaces(s) and for at least two persons using the same meeting space(s) to see each other through the meeting space(s).

In some embodiments, the means comprise one or more of the following:
a) at least one heat source and/or heated air produced by a heat source and/or a thermal updraft;
b) ultraviolet and/or infrared and/or concentrated solar radiation;
c) at least one electrostatic attractor and/or at least one kind of airborne ion;
d) at least one anti-pathogenic agent;
e) at least one airflow.

In some embodiments, the invention is a plurality of systems providing more than one safe meeting space for two or more persons, wherein each system comprises the system as described elsewhere here.

In some embodiments, the invention is a plurality of systems providing more than one safe meeting space for two or more persons, wherein each system is an embodiment of the invention.

In some embodiments, the invention is a system providing one or more safe meeting space for two or more persons wherein:

a1) the system provides a central meeting space and person locations outside the central meeting space for persons using the central meeting space, with no barriers between the central meeting space and the person locations such that persons in the person locations can communicate with each other through the central meeting space, and outside the central meeting space and between adjacent person locations, are provided adjacent person meeting spaces or partitions, or

a2) the system provides more than one meeting space and person locations outside the meeting spaces for persons using the meeting spaces, with no barriers between the meeting space(s) and the person locations such that persons in the person locations using the same meeting space(s) can communicate with each other through the meeting space(s),

b) the system is configured such that substantially all pathways within the system for the possible spread of pathogens between the person locations pass through at least one of the meeting spaces and/or the system is configured such that substantially all pathways within the system for the possible spread of pathogens between persons using the same meeting spaces pass through at least one of the meeting spaces,

c) means are provided to make the meeting space(s) sterile or to substantially reduce the number of pathogens in the meeting spaces(s),

d) the system is configured to allow for persons in the person locations using the same meeting space(s) to hear each other through the meeting spaces(s) and for at least two persons using the same meeting space(s) to see each other through the meeting space(s).

In some embodiments, the means comprise one or more of the following:

a) at least one heat source and/or heated air and/or a thermal updraft;

b) ultraviolet and/or infrared and/or concentrated solar radiation;

c) at least one electrostatic attractor and/or at least one kind of airborne ion;

d) at least one anti-pathogenic agent; and

e) at least one airflow.

In some embodiments, the means utilize solar energy and/or stored solar energy and comprise one or more of the following:

a) UV extracted from sunlight;

b) IR extracted from sunlight;

c) concentrated sunlight;

d) solar thermal energy;

e) a thermal updraft created by solar energy; and

f) an electrostatic field and/or airborne ions and/or ozone produced by solar energy.

In some embodiments, the means is at least one electrostatic attractor and/or at least one kind of airborne ion; so as to provide one or more airborne ion that binds to pathogens and inactivates them, and/or one or more differently charged electrostatic attractor which extracts pathogens from the meeting space(s) and/or draws pathogens away from persons, and/or the electrostatic attractor draws ions through the meeting space(s) and/or moves them away from persons, and/or the electrostatic attractor extracts pathogens bound to ions from the meeting space(s) and/or moves pathogens bound to ions away from persons.

In some embodiments, the system provides more than one safe meeting space for two or more persons comprising one or more systems (each of which are embodiments) and further providing means to prevent pathogens passing between persons using different meeting spaces.

In some embodiments, the means for the adjacent person space(s) and/or for meeting spaces that are close to persons are not harmful to the persons.

In some embodiments, the means that are not harmful to persons comprise one or more of the following:

a) airflows and/or air extraction means;

b) heated air of sufficient temperature to destroy pathogens and/or to extract pathogens; by creating a convective updraft, without being of sufficient temperature to harm persons;

c) safe UV C;

d) an electrostatic attractor and/or at least one kind of airborne ion; and

e) safe levels of solar IR and/or solar UV and/or concentrated sunlight for brief exposure.

In some embodiments, heat energy that provides the means and/or heated air from one or more meeting spaces is also used to heat buildings or outdoor spaces.

In some embodiments, heated air from the meeting space(s) is fed back into the meeting space(s).

In some embodiments, pathogens are destroyed/removed from the heated air before it is used to heat buildings/outdoor spaces.

In some embodiments, pathogens are destroyed/removed from the heated air before it is fed back into the meeting space(s).

In some embodiments, heat energy provided by the means and/or heat energy from one or more meeting spaces is used to destroy/remove pathogens from the space in which the system is located.

In some embodiments, the meeting space(s) have air extraction means so that the meeting spaces are at negative pressure in relation to the person locations and/or the meeting space(s) are at negative pressure in relation to the space in which the system is located.

Some embodiments, further provide clean airflows to the person locations and/or to the zone surrounding person locations so that the person locations and/or the zone surrounding person locations are at positive pressure in relation to the meeting space(s).

In some embodiments, the means further comprise means to create a pleasing visual image which serves as a focus for conversation and/or to display a subject for discussion in the meeting space(s).

Some embodiments, further comprise sound reflectors to enhance communication between the persons.

Some embodiments include a method of using the inventive system to safely accommodate one or more of meetings, face-to-face conversation, discussion, networking events, business meetings, focus groups, conferences, conventions, discussion forums, discussion groups and educational activities, social events involving conversation or discussion between persons.

Some embodiments include a method of using the inventive system, wherein the one or more meeting space is used for one or more of the following:

a) advertising or promotional or sales events and/or advertising or promotional or sales events that involve face-to-face conversation

b) meetings for the provision of business services or customer services;

c) events for the provision of financial services;

d) focus groups, and optionally creating focus groups for market research or for product development or for political organizations;

e) political events;

f) cultural events;

g) conventions;

h) book fairs;

i) trade shows;

j) community events;

k) civic events;

l) personal development events;

m) professional development events;

n) improving communication within companies or organizations;

o) improving communication between companies or between organizations;

p) group therapy or other therapeutic use based on face-to-face conversation;

q) medical or therapeutic use by providing social interaction;

r) medical or therapeutic use by providing social connection;

s) medical or therapeutic use by connecting patient groups with related medical conditions;

t) medical or therapeutic use by sharing experiences of medical conditions,

u) educational use to teach or learn communication skills or networking skills or social skills;

v) educational use that involves teaching or learning by face-to-face discussion

w) recruitment events;

x) matching job applicants to employers;

y) networking between job applicants and employers;

z) mediation events, mediation between groups or individuals;

aa) conflict resolution events;

bb) dating events,

cc) networking events arising from conferences or conventions; and networking events arising from conferences or conventions involving the creation of breakout groups,

dd) business networking,

ee) social networking,

ff) professional career networking,

gg) expert networking,

hh) expert networking in areas including medicine and related professions, science, biotechnology, pharmaceuticals, technology, engineering, computing, electronics, digital technology, and

ii) a musical performance.

I claim:

1. A system providing one or more safe meeting space for two or more persons wherein:
a1) the system provides a central meeting space and person locations outside the central meeting space for persons using the central meeting space, with no barriers between the central meeting space and the person locations such that persons in the person locations can communicate with each other through the central meeting space, and outside the central meeting space and between adjacent person locations, are provided adjacent person meeting spaces or partitions, or
a2) the system provides more than one meeting space and person locations outside the meeting spaces for persons using the meeting spaces, with no barriers between the meeting space(s) and the person locations such that persons in the person locations using the same meeting space(s) can communicate with each other through the meeting space(s),
b) the system is configured such that substantially all pathways within the system for the possible spread of pathogens between the person locations pass through at least one of the meeting spaces and/or the system is configured such that substantially all pathways within the system for the possible spread of pathogens between persons using the same meeting spaces pass through at least one of the meeting spaces,
c) means are provided to make the meeting space(s) sterile or to substantially reduce the number of pathogens in the meeting spaces(s),
d) the system is configured to allow for persons in the person locations using the same meeting space(s) to hear each other through the meeting spaces(s) and for at least two persons using the same meeting space(s) to see each other through the meeting space(s).

2. The system of claim 1 wherein the means comprise one or more of the following:
a) at least one heat source and/or heated air and/or a thermal updraft;
b) ultraviolet and/or infrared and/or concentrated solar radiation;
c) at least one electrostatic attractor and/or at least one kind of airborne ion;
d) at least one anti-pathogenic agent; and
e) at least one airflow.

3. The system of claim 2 wherein the means utilize solar energy and/or stored solar energy and comprise one or more of the following:
g) UV extracted from sunlight;
h) IR extracted from sunlight;
i) concentrated sunlight;
j) solar thermal energy;
k) a thermal updraft created by solar energy; and
l) an electrostatic field and/or airborne ions and/or ozone produced by solar energy.

4. The system of claim 2 wherein the means is at least one electrostatic attractor and/or at least one kind of airborne ion; so as to provide one or more airborne ion that binds to pathogens and inactivates them, and/or one or more differently charged electrostatic attractor which extracts pathogens from the meeting space(s) and/or draws pathogens away from persons, and/or the electrostatic attractor draws ions through the meeting space(s) and/or moves them away from persons, and/or the electrostatic attractor extracts pathogens bound to ions from the meeting space(s) and/or moves pathogens bound to ions away from persons.

5. A system providing more than one safe meeting space for two or more persons comprising one or more systems of claim 1 and further providing means to prevent pathogens passing between persons using different meeting spaces.

6. The system of claim 1, wherein the means for the adjacent person space(s) and/or for meeting spaces that are close to persons are not harmful to the persons.

7. The system of claim 6 wherein the means that are not harmful to persons comprise one or more of the following:
a) airflows and/or air extraction means;
b) heated air of sufficient temperature to destroy pathogens and/or to extract pathogens; by creating a convective updraft, without being of sufficient temperature to harm persons;
c) safe UV C;
d) an electrostatic attractor and/or at least one kind of airborne ion; and
e) safe levels of solar IR and/or solar UV and/or concentrated sunlight for brief exposure.

8. The system of claim 3, wherein heat energy that provides the means and/or heated air from one or more meeting spaces is also used to heat buildings or outdoor spaces.

9. The system of claim 3, wherein heated air from the meeting space(s) is fed back into the meeting space(s).

10. The system of claim 8, wherein pathogens are destroyed/removed from the heated air before it is used to heat buildings/outdoor spaces.

11. The system of claim 9 wherein pathogens are destroyed/removed from the heated air before it is fed back into the meeting space(s).

12. The system of claim 1, wherein heat energy provided by the means and/or heat energy from one or more meeting spaces is used to destroy/remove pathogens from the space in which the system is located.

13. The system of claim 1 wherein the meeting space(s) have air extraction means so that the meeting spaces are at negative pressure in relation to the person locations and/or the meeting space(s) are at negative pressure in relation to the space in which the system is located.

14. The system of claim 1 further providing clean airflows to the person locations and/or to the zone surrounding person locations so that the person locations and/or the zone surrounding person locations are at positive pressure in relation to the meeting space(s).

15. The system of claim 1 wherein the means further comprise means to create a pleasing visual image which serves as a focus for conversation and/or to display a subject for discussion in the meeting space(s).

16. The system of claim 1, further comprising sound reflectors to enhance communication between the persons.

17. A method of using the system as claimed in claim 1 to safely accommodate one or more of meetings, face-to-face conversation, discussion, networking events, business meetings, focus groups, conferences, conventions, discussion forums, discussion groups and educational activities, social events involving conversation or discussion between persons.

18. A method of using the system of claim 1, wherein the one or more meeting space is used for one or more of the following:
jj) advertising or promotional or sales events and/or advertising or promotional or sales events that involve face-to-face conversation
kk) meetings for the provision of business services or customer services;
ll) events for the provision of financial services;
mm) focus groups, and optionally creating focus groups for market research or for product development or for political organizations;
nn) political events;
oo) cultural events;
pp) conventions;
qq) book fairs;
rr) trade shows;
ss) community events;
tt) civic events;
uu) personal development events;
vv) professional development events;
ww) improving communication within companies or organizations;
xx) improving communication between companies or between organizations;
yy) group therapy or other therapeutic use based on face-to-face conversation;
zz) medical or therapeutic use by providing social interaction;
aaa) medical or therapeutic use by providing social connection;
bbb) medical or therapeutic use by connecting patient groups with related medical conditions;
ccc) medical or therapeutic use by sharing experiences of medical conditions,
ddd) educational use to teach or learn communication skills or networking skills or social skills;
eee) educational use that involves teaching or learning by face-to-face discussion
fff) recruitment events;
ggg) matching job applicants to employers;
hhh) networking between job applicants and employers;
iii) mediation events, mediation between groups or individuals;
jjj) conflict resolution events;
kkk) dating events,
lll) networking events arising from conferences or conventions; and networking events arising from conferences or conventions involving the creation of breakout groups,
mmm) business networking,
nnn) social networking,
ooo) professional career networking,
ppp) expert networking,
qqq) expert networking in areas including medicine and related professions, science, biotechnology, pharmaceuticals, technology, engineering, computing, electronics, digital technology, and
rrr) a musical performance.
